SEQUENCE LISTING (83 sequences)

SEQ ID NO: 1 - DNA, 417 bp, Artificial Sequence
  Synthetic Heavy chain DNA sequence
atggctgtcctggcactactcctctgcctggtggctttcccaagctgtaccctgtcccaggtgcaactgaaggagtcaggacctggcctggtggcgccctcacagagcctatccattacctgcactgtctctggattctcattaaccacctatgatataaactggattcgccagccaccaggaaagggtctggagtggcttggaataatatggactggtggaggcacaaattataattcagctttcatgtccagactgagcatcagcaaggacaactccaagagccaagttttcttaaaaatgaacagtctgcaaactgatgacacagccatatattactgtgtaagagatcggggttactacgtttactattctatggactactggggtcaaggaacctcagtcaccgtctcctca

SEQ ID NO: 2 - protein, 139 aa, Artificial Sequence
  Synthetic Heavy chain amino acid sequence
MAVLALLLCLVAFPSCTLSQVQLKESGPGLVAPSQSLSITCTVSGFSLTTYDINWIRQPPGKGLEWLGIIWTGGGTNYNSAFMSRLSISKDNSKSQVFLKMNSLQTDDTAIYYCVRDRGYYVYYSMDYWGQGTSVTVSS

SEQ ID NO: 3 - DNA, 381 bp, Artificial Sequence
  Synthetic Light chain DNA sequence
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatatgtttccgtggacgttcggtggaggcaccaagctggaaatcaaa

SEQ ID NO: 4 - protein, 127 aa, Artificial Sequence
  Synthetic Light chain amino acids sequence
MMSSAQFLGLLLLCFQGTRCDIQMTQTTSSLSASLGDRVTISCRASQDISNYLNWYQQKPDGTVKLLIYYTSRLHSGVPSRFSGSGSGTDYSLTISNLEQEDIATYFCQQGNMFPWTFGGGTKLEIK

SEQ ID NO: 5 - protein, 10 aa, Artificial Sequence
  Synthetic Heavy chain CDR1
GFSLTTYDIN

SEQ ID NO: 6 - protein, 16 aa, Artificial Sequence
  Synthetic Heavy chain CDR2
IIWTGGGTNYNSAFMS

SEQ ID NO: 7 - protein, 12 aa, Artificial Sequence
  Synthetic Heavy chain CDR3
DRGYYVYYSMDY

SEQ ID NO: 8 - protein, 11 aa, Artificial Sequence
  Synthetic Light chain CDR1
RASQDISNYLN

SEQ ID NO: 9 - protein, 7 aa, Artificial Sequence
  Synthetic Light chain CDR2
YTSRLHS

SEQ ID NO: 10 - protein, 9 aa, Artificial Sequence
  Synthetic Light chain CDR3
QQGNMFPWT

SEQ ID NO: 11 - DNA, 546 bp, Artificial Sequence
  Synthetic Truncated C-terminal of CPa, ORF
tcaaaagaatatgcaagaggttttgctaaaacaggaaaatcaatatactatagtcatgctagcatgagtcatagttgggatgattgggactatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgaaaaagatgctggaacagatgactacatgtattttggaatcaaaacaaaggatggaaaaactcaagaatgggaaatggacaacccaggaaatgattttatgactggaagtaaagatacttatactttcaaattaaaagatgaaaatctaaaaattgatgatatacaaaatatgtggattagaaaaagaaaatatacagcattcccagatgcttataagccagaaaacataaaggtaatagcaaatggaaaagttgtagtggacaaggatataaatgagtggatttcaggaaattcaacttataatataaaa

SEQ ID NO: 12 - protein, 182 aa, Artificial Sequence
  Synthetic Translated
SKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKVIANGKVVVDKDINEWISGNSTYNIK

SEQ ID NO: 13 - DNA, 792 bp, Artificial Sequence
  Synthetic DAG1, ORF
atgacacagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccattagcaatgtgcagtctgaagacttggcagattatttctgtcagcaatatagcagctatcctctcacgttcggaggggggaccaagctggaaataaaaggtggttcctctagatcttccctcgaggttcagctgcagcagtctggggctgaactggtgaagcctgggacttcagtgaggatatcctgcaaggcttctggctacaccttcacaaactactatatatactgggtgaagcagaggcctggacagggacttgagtggattggatggatttatcctggaaatgttaatactaagtacaatgagaagttcaagggcaaggccacactgactgtagacaaatcctccagcacagcctacatgcagctcagcagcctgacctctgaggactctgcggtctatttctgtgcaagaagggggactgggacggtcgtttttgactactggggccacggcaccactcttacagtctcctcagccaaaacaacacccccatctgtcactagtggccaggccggccagcaccatcaccatcaccatggcgcatacccgtacgacgttccggactacgcttct

SEQ ID NO: 14 - protein, 264 aa, Artificial Sequence
  Synthetic Translated
MTQSQKFMSTSVGDRVSVTCKASQNVGTNVAWYQQKPGQSPKALIYSASYRYSGVPDRFTGSGSGTDFTLTISNVQSEDLADYFCQQYSSYPLTFGGGTKLEIKGGSSRSSLEVQLQQSGAELVKPGTSVRISCKASGYTFTNYYIYWVKQRPGQGLEWIGWIYPGNVNTKYNEKFKGKATLTVDKSSSTAYMQLSSLTSEDSAVYFCARRGTGTVVFDYWGHGTTLTVSSAKTTPPSVTSGQAGQHHHHHHGAYPYDVPDYAS

SEQ ID NO: 15 - DNA, 1383 bp, Artificial Sequence
  Synthetic DAG1-Cpa
atgacacagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccattagcaatgtgcagtctgaagacttggcagattatttctgtcagcaatatagcagctatcctctcacgttcggaggggggaccaagctggaaataaaaggtggttcctctagatcttccctcgaggttcagctgcagcagtctggggctgaactggtgaagcctgggacttcagtgaggatatcctgcaaggcttctggctacaccttcacaaactactatatatactgggtgaagcagaggcctggacagggacttgagtggattggatggatttatcctggaaatgttaatactaagtacaatgagaagttcaagggcaaggccacactgactgtagacaaatcctccagcacagcctacatgcagctcagcagcctgacctctgaggactctgcggtctatttctgtgcaagaagggggactgggacggtcgtttttgactactggggccacggcaccactcttacagtctcctcagccaaaacaacacccccatctgtcactagtggccaggccggccagcaccatcaccatcaccatggcgcatacccgtacgacgttccggactacgcttctggtggaggcggttcaggcggaggtggctctggcggtggcggatcatcaaaagaatatgcaagaggttttgctaaaacaggaaaatcaatatactatagtcatgctagcatgagtcatagttgggatgattgggactatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgaaaaagatgctggaacagatgactacatgtattttggaatcaaaacaaaggatggaaaaactcaagaatgggaaatggacaacccaggaaatgattttatgactggaagtaaagatacttatactttcaaattaaaagatgaaaatctaaaaattgatgatatacaaaatatgtggattagaaaaagaaaatatacagcattcccagatgcttataagccagaaaacataaaggtaatagcaaatggaaaagttgtagtggacaaggatataaatgagtggatttcaggaaattcaacttataatataaaa

SEQ ID NO: 16 - protein, 461 aa, Artificial Sequence
  Synthetic Translated (461aa) -DAG1, (G4S)3, C-terminal of Cpa
MTQSQKFMSTSVGDRVSVTCKASQNVGTNVAWYQQKPGQSPKALIYSASYRYSGVPDRFTGSGSGTDFTLTISNVQSEDLADYFCQQYSSYPLTFGGGTKLEIKGGSSRSSLEVQLQQSGAELVKPGTSVRISCKASGYTFTNYYIYWVKQRPGQGLEWIGWIYPGNVNTKYNEKFKGKATLTVDKSSSTAYMQLSSLTSEDSAVYFCARRGTGTVVFDYWGHGTTLTVSSAKTTPPSVTSGQAGQHHHHHHGAYPYDVPDYASGGGGSGGGGSGGGGSSKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKVIANGKVVVDKDINEWISGNSTYNIK

SEQ ID NO: 17 - protein, 11 aa, Artificial Sequence
  Synthetic VL CDR1
KASQNVGTNVA

SEQ ID NO: 18 - protein, 7 aa, Artificial Sequence
  Synthetic VL CDR2
SASYRYS

SEQ ID NO: 19 - protein, 9 aa, Artificial Sequence
  Synthetic VL CDR3
QQYSSYPLT

SEQ ID NO: 20 - protein, 5 aa, Artificial Sequence
  Synthetic VH CDR1
NYYIY

SEQ ID NO: 21 - protein, 17 aa, Artificial Sequence
  Synthetic VH CDR2
WIYPGNVNTKYNEKFKG

SEQ ID NO: 22 - protein, 11 aa, Artificial Sequence
  Synthetic VH CDR3
RGTGTVVFDYW

SEQ ID NO: 23 - protein, 6 aa, Artificial Sequence
  Synthetic His Tag
HHHHHH

SEQ ID NO: 24 - protein, 12 aa, Artificial Sequence
  Synthetic HA Tag
GAYPYDVPDYAS

SEQ ID NO: 25 - protein, 6 aa, Artificial Sequence
  Synthetic protective epitope of CPa
ARGFAK

SEQ ID NO: 26 - protein, 8 aa, Avian Influenza virus
  misc_feature (1)..(8): m2e
EVETPIRN

SEQ ID NO: 27 - protein, 8 aa, Avian Influenza virus
  misc_feature (1)..(8): m2e
EVETPTRN

SEQ ID NO: 28 - protein, 12 aa, Avian Influenza virus
  misc_feature (1)..(12): HA5 UA
LLSRINHFEKIQ

SEQ ID NO: 29 - protein, 19 aa, Avian Influenza virus
  misc_feature (1)..(19): HA5 LB
ANPANDLCYPGDFNDYEEL

SEQ ID NO: 30 - protein, 16 aa, Avian Influenza virus
  misc_feature (1)..(16): NP 54-69
GRLIQNSITIERMVLS

SEQ ID NO: 31 - protein, 14 aa, Avian Influenza virus
  misc_feature (1)..(14): NP 147-160
TYQRTRALVRTGMD

SEQ ID NO: 32 - protein, 19 aa, E. coli
  misc_feature (1)..(19): PAL bis from E. coli
EGHADERGTPEYNISLGER

SEQ ID NO: 33 - protein, 6 aa, Artificial Sequence
  Synthetic
GHADER

SEQ ID NO: 34 - protein, 6 aa, Artificial Sequence
  Synthetic
DERGTP

SEQ ID NO: 35 - protein, 6 aa, Artificial Sequence
  Synthetic
EYNISL

SEQ ID NO: 36 - protein, 6 aa, Artificial Sequence
  Synthetic
ISLGER

SEQ ID NO: 37 - protein, 19 aa, Vibrio spp.
  misc_feature (1)..(19): PALbis from vibrio spp.
EGHADERGTPEYNIALGER

SEQ ID NO: 38 - protein, 17 aa, Campylobacter spp.
  misc_feature (1)..(17): corresponding peptide from Campylobacter spp.
EGNCDEWGTDEYNQALG

SEQ ID NO: 39 - protein, 19 aa, E. coli
  misc_feature (1)..(19): PAL  from E. coli
TVEGHADERGTPEYNISLG

SEQ ID NO: 40 - protein, 21 aa, Campylobacter jejuni
  misc_feature (1)..(21): Campylobacter jejuni Cj0113
GVSITVEGNCDEWGTDEYNQA

SEQ ID NO: 41 - protein, 19 aa, Vibrio spp.
  misc_feature (1)..(19): Vibrio spp. alternative PAL epitope
TVEGHADERGTPEYNIALG

SEQ ID NO: 42 - DNA, 57 bp, E. coli
  misc_feature (1)..(57): E. coli nucleotide sequence for PAL epitope
gaaggtcacgcggacgaacgtggtaccccggaatacaacatctctctgggtgaacgt

SEQ ID NO: 43 - protein, 9 aa, E. coli
  misc_feature (1)..(9): Epitope of PAL from E. coli
EYNISLGER

SEQ ID NO: 44 - protein, 9 aa, Vibrio spp.
  misc_feature (1)..(9): Epitope of PAL from Vibrio spp.
EYNIALGER

SEQ ID NO: 45 - protein, 16 aa, Artificial Sequence
  Synthetic composite minimal epitope
PXXXXXGYGACEXNLG

SEQ ID NO: 46 - protein, 43 aa, Eimeria maxima
  misc_feature (1)..(43): MPP
PSHDAPESERTPRVISFGYGACEHNLGVSLFRREETKKDPRGR

SEQ ID NO: 47 - protein, 28 aa, Neospora canium
PRIVSFGYGACEHNLGMSLYDRQGLQRQ

SEQ ID NO: 48 - protein, 21 aa, Eimeria tenella
ESQRAPMVIRYGYGACEYNLG

SEQ ID NO: 49 - protein, 10 aa, Eimeria maxima
  misc_feature (1)..(10): TRAP-1
GGGFPTAAVA

SEQ ID NO: 50 - protein, 40 aa, Eimeria maxima
  misc_feature (1)..(40): TRAP-02
AAPETPAVQPKPEEGHERPEPEEEEEKKEEGGGFPTAAVA

SEQ ID NO: 51 - protein, 40 aa, Eimeria maxima
  misc_feature (1)..(40): TRAP-03
GGGFPTAAVAGGVGGVLLIAAVGGGVAAFTSGGGGAGAQE

SEQ ID NO: 52 - protein, 21 aa, Campylobacter jejuni
  misc_feature (1)..(21): Cj0982
KDIVLDAEIGGVAKGKDGKEK

SEQ ID NO: 53 - protein, 35 aa, Campylobacter jejuni
  misc_feature (1)..(35): Cj0420
KVALGVAVPKDSNITSVEDLKDKTLLLNKGTTADA

SEQ ID NO: 54 - protein, 276 aa, Gallus gallus
  misc_feature (1)..(276): CD40
MGRLGLLGLLCALLLGCGQPGDAVNCSDKQYEHKGRCCNRCQPGKKLASECNDTEDSVCTPCENGQYQQSWTKERHCTPHEICEDNAGLIVKRHGNATHNTVCQCRAGMHCSDASCQTCVENEPCKQGFGFVAAMAEARMTSPCEPCAEGTFSNVSSKTEPCHFWTSCEEKGLVVKVKGTNTSDVICESSRRSSLSVLIPITAAVVTCLVGICIYCLVHTDLRRRGPKQAEAEAPRELVTQQPEEVDFPVQETRLGGQPVAQEDGKESRIAEQEQL

SEQ ID NO: 55 - protein, 280 aa, Bos Taurus
  misc_feature (1)..(280): CD40
MVRLPLQCLFWGFFLTAVHSEPATACGEKQYPVNSLCCDLCPPGQKLVNDCTEVSKTECQSCGKGEFLSTWNREKYCHEHRYCNPNLGLRIQSEGTLNTDTTCVCVEGQHCTSHTCESCTPHSLCLPGFGVKQIATGLLDTVCEPCPLGFFSNVSSAFEKCHRWTSCERKGLVEQHVGTNKTDVVCGFQSRMRTLVVIPVTMGVLFAVLLVSACIRNITKKRQAKALHPTAERQDPVETIDPEDFPGPHPPPPVQETLCWCQPVAQEDGKESRISVQERE

SEQ ID NO: 56 - protein, 278 aa, Sus scrofa domesticus
  misc_feature (1)..(278): CD40
MVRLPLKCLLWGCFLTAVHPEPPTSCKENQYPTNSRCCNLCPPGQKLVNHCTEVTETECLPCSSSEFLATWNREKHCHQHKYCDPNLGLQVQREGTSKTDTTCVCSEGHHCTNSACESCTLHSLCFPGLGVKQMATEVSDTICEPCPVGFFSNVSSASEKCQPWTSCESKGLVEQRAGTNKTDVVCGFQSRMRALVVIPITLGILFAVLLVFLCIRKVTKEQETKALHPKTERQDPVETIDLEDFPDSTAPVQETLHWCQPVTQEDGKESRISVQERE

SEQ ID NO: 57 - protein, 16 aa, Clostridium perfringens
  misc_feature (1)..(16): Alpha toxin
NAWSKEYARGFAKTGK

SEQ ID NO: 58 - protein, 9 aa, Avian influenza
  misc_feature (1)..(9): M2e peptide
CEVETPTRN

SEQ ID NO: 59 - protein, 9 aa, Clostridium perfringens
  misc_feature (1)..(9): Alpha-31
GKIDGTGTH

SEQ ID NO: 60 - protein, 15 aa, Clostridium perfringens
  misc_feature (1)..(15): Alpha-51
ENDMSKNEPESVRKN

SEQ ID NO: 61 - protein, 20 aa, Clostridium perfringens
  misc_feature (1)..(20): Alpha-71
ENMHELQLGSTYPDYDKNAY

SEQ ID NO: 62 - protein, 20 aa, Clostridium perfringens
  misc_feature (1)..(20): Alpha-81
TYPDYDKNAYDLYQDHFWDP

SEQ ID NO: 63 - protein, 20 aa, Clostridium perfringens
  misc_feature (1)..(20): Alpha-91
DLYQDHFWDPDTDNNFSKDN

SEQ ID NO: 64 - protein, 10 aa, Clostridium perfringens
  misc_feature (1)..(10): Alpha-117
IPDTGESQIR

SEQ ID NO: 65 - protein, 10 aa, Clostridium perfringens
  misc_feature (1)..(10): Alpha-136
EWQRGNYKQA

SEQ ID NO: 66 - protein, 23 aa, Clostridium perfringens
  misc_feature (1)..(23): Alpha-158
DIDTPYHPANVTAVDSAGHVKFE

SEQ ID NO: 67 - protein, 20 aa, Clostridium perfringens
  misc_feature (1)..(20): Alpha-170
VDSAGHVKFETFAEERKEQY

SEQ ID NO: 68 - protein, 20 aa, Clostridium perfringens
  misc_feature (1)..(20): Alpha-181
TFAEERKEQYKINTAGCKTN

SEQ ID NO: 69 - protein, 21 aa, Clostridium perfringens
  misc_feature (1)..(21): Alpha-191
KINTVGCKTNEDFYADILKNK

SEQ ID NO: 70 - protein, 20 aa, Clostridium perfringens
  misc_feature (1)..(20): Alpha-200
EDFYADILKNKDFNAWSKEY

SEQ ID NO: 71 - protein, 20 aa, Clostridium perfringens
  misc_feature (1)..(20): Alpha-210
KDFNAWSKEYARGFAKTGKS

SEQ ID NO: 72 - protein, 17 aa, Clostridium perfringens
  misc_feature (1)..(17): Alpha-220
ARGFAKTGKSIYYSHAS

SEQ ID NO: 73 - protein, 17 aa, Clostridium perfringens
  misc_feature (1)..(17): Alpha-233
SHASMSHSWDDWDYAAK

SEQ ID NO: 74 - protein, 20 aa, Clostridium perfringens
  misc_feature (1)..(20): Alpha-240
SWDDWDYAAKVTLANSQKGT

SEQ ID NO: 75 - protein, 16 aa, Clostridium perfringens
  misc_feature (1)..(16): Alpha-270
DVSEGNDPSVGNNVKE

SEQ ID NO: 76 - protein, 12 aa, Clostridium perfringens
  misc_feature (1)..(12): Alpha-291
STSGEKDAGTDD

SEQ ID NO: 77 - protein, 13 aa, Clostridium perfringens
  misc_feature (1)..(13): Alpha-309
KTKDGKTQEWEMD

SEQ ID NO: 78 - protein, 21 aa, Clostridium perfringens
  misc_feature (1)..(21): Alpha-320
DNPGNDFMAGSKDTYTFKLKD

SEQ ID NO: 79 - protein, 20 aa, Clostridium perfringens
  misc_feature (1)..(20): Alpha-330
SKDTYTFKLKDENLKIDDIQ

SEQ ID NO: 80 - protein, 16 aa, Clostridium perfringens
  misc_feature (1)..(16): Alpha-354
RKRKYTAFPDAYKPEN

SEQ ID NO: 81 - protein, 19 aa, Clostridium perfringens
  misc_feature (1)..(19): Alpha-379
VVDKDINEWISGNSTYNIK

SEQ ID NO: 82 - protein, 20 aa, Artificial Sequence
  Synthetic Alpha-210 Control
KDFNAWSKEYARGFAKTGKS

SEQ ID NO: 83 - protein, 17 aa, Artificial Sequence
  Synthetic Alpha-220 Control
ARGFAKTGKSIYYSHAS

(19) United States
(12) Patent Application Publication
(10) Pub. No.: US 2017/0196971 A1
(43) Pub. Date: Jul. 13, 2017

(54) ANTIBODY GUIDED VACCINES AND METHODS OF USE FOR GENERATION OF RAPID MATURE IMMUNE RESPONSES

(71) Applicants: THE BOARD OF TRUSTEES OF THE UNIVERSITY OF ARKANSAS, Little Rock, AR (US); THE TEXAS A&M UNIVERSITY SYSTEM, College Station, TX (US)

(72) Inventors: Luc R. Berghman, College Station, TX (US); Daad Abi-Ghanem, Tigard, OR (US); Chang-Hsin Chen, College Station, TX (US); Wen-Ko Chou, College Station, TX (US); Christine Vuong, Bryan, TX (US); Suryakant Waghela, College Station, TX (US); Waithaka Mwangi, College Station, TX (US); Billy Hargis, Fayetteville, AR (US); Lisa Bielke, Wooster, OH (US)

(73) Assignees: THE BOARD OF TRUSTEES OF THE UNIVERSITY OF ARKANSAS, Little Rock, AR (US); THE TEXAS A&M UNIVERSITY SYSTEM, College Station, TX (US)

(21) Appl. No.: 15/316,421

(22) PCT Filed: Jun. 4, 2015
(86) PCT No.: PCT/US15/34229

§ 371 Date: Dec. 5, 2016

Related U.S. Application Data
(60) Provisional application No. 62/008,178, filed on Jun. 5, 2014.

(51) Int. Cl. A61K 39/39; G01N 33/68; A61K 39/145; C07K 16/28; C07K 16/10

(52) U.S. Cl. CPC A61K 39/39; C07K 16/2878; C07K 16/1018; A61K 39/145; G01N 33/6878; C07K 2317/75; C07K 2317/55; C07K 2317/565; A61K 2039/5252

(57) ABSTRACT

Adjuvant compositions, vaccines, constructs for preparing the adjuvant compositions and vaccines and methods of using the adjuvant compositions and vaccines to enhance immune responses in subjects are provided herein. In particular, a rapid antibody response to the vaccine including both IgG (in the circulation) and sIgA (mucosal secretory IgA) is elicited. The adjuvants and vaccines may be used for sub-cutaneous or mucosal administration enabling low cost, effective vaccination of subjects. A method of epitope mapping to rapidly identify antigenic epitopes is also provided.

### CROSS-REFERENCE TO RELATED APPLICATIONS

This patent application claims the benefit of priority of U.S. Provisional Patent Application No. 62/008,178, filed Jun. 5, 2014, which is incorporated herein by reference in its entirety.

### STATEMENT REGARDING FEDERALLY SPONSORED RESEARCH

This invention was made with United States government support awarded by the National Institute of Food and Agriculture grant number 2008-35204-04554. The United States has certain rights in this invention.

### SEQUENCE LISTING

This application includes an electronically submitted Sequence Listing in .txt format. The .txt file contains a sequence listing entitled “2015-05-29_5658-00264_ST25.txt” created on May 31, 2015 and is 43,879 bytes in size. The Sequence Listing contained in this .txt file is part of the specification and is hereby incorporated by reference herein in its entirety.

### INTRODUCTION

Mucosal surfaces are vast surface areas that are the major portal of entrance of a wide range of pathogens. Therefore, the mediation of adaptive immunity at the mucosal sites is a key objective for improving vaccine efficacy. A means of inducing rapid mucosal immune responses in response to vaccination is needed.

Vaccination has the great potential to be a vehicle to deliver antigen and induce an antigen-specific adaptive immune response in mucosal sites. However, direct mucosal immunization has been found to be difficult due to several factors including dilution of mucosal vaccines in the bulk of mucosal fluid that limits absorption of antigen by the mucosal epithelium. Due to the complexity of mucosal surfaces, mucosal vaccines frequently fail to transverse the mucosal gel and are subsequently degraded by proteases.

Several mucosal vaccines are universally used in poultry industry. However, most of these mucosal vaccines can only induce a local IgA immune response, and they are unable to react against the pathogen once it spreads through the circulation. Thus, a new formulation of vaccines that is capable of inducing both local mucosal and systemic immune responses is desired. The goal of any mucosal vaccine design is to increase immunogenicity (useful effector mechanisms) without leading to reactogenicity (inflammation, hypersensitivity, etc.). Among the various strategies under development, there is great potential for novel vaccines based on recombinant, proteins and synthetic peptides. However, such antigens often lack the immunogenicity of live attenuated or whole killed pathogens used in traditional vaccines. There is, therefore, an urgent need to develop immunological adjuvants with a high potential to enhance immune responses while simultaneously possessing a low potential of negative side effects.

A number of mucosal adjuvants for co-administration with live attenuated vaccines through the oculo-nasal or oral routes have been reported in chickens. Despite the fact that some of these adjuvants do enhance mucosal sIgA and systemic IgG responses, they are still considered time- and antigen-consuming since repeated injections of a large amount of antigen are still required.

### SUMMARY

Provided herein are adjuvants vaccines, constructs for preparing the adjuvants and vaccines and methods of using the adjuvants and vaccines to enhance immune responses in subjects. In particular a rapid antibody response to the vaccine including both IgG (in the circulation) and sIgA (mucosal secretory IgA) is elicited. The adjuvants and vaccines may be used for sub-cutaneous of mucosal administration enabling low cost, effective vaccination of subjects.

In one aspect, an adjuvant composition comprising a first CD40 agonistic antibody or portion thereof comprising at least two F(ab) regions capable of specifically binding CD40 and inducing CD40 signaling, at least one second antibody or portion thereof comprising at least two F(ab) regions capable of specifically binding a microorganism, at least one label attached to the at least one first CD40 agonistic antibody or portion thereof and the at least one second antibody or portion thereof, and a linker moiety capable of specifically binding to the labels with high affinity. The first CD40 agonistic antibody and the second antibody are bound to the linker moiety to form a complex. The second antibody may be capable of binding a microorganism that may include a virus, bacterium, vaccine vector, killed pathogen or parts thereof. The second antibody may be specific for an epitope on the surface of the microorganism. The epitope may be conserved. The CD40 agonistic antibody may be specific for chicken CD40 and may include or consist of SEQ ID NO: 2 and SEQ ID NO: 4 or SEQ ID NO: 14. Alternatively the CD40 agonistic antibody may include the CDR regions of SEQ ID NOs: 5-10 or the CDR regions of SEQ NOs: 17-22. The killed pathogen may be Influenza or a bacterium or a bacterial cell surface fragment.

The adjuvant composition can be combined with the microorganism via interaction with the second antibody to produce a vaccine. The serotype of the microorganism may be unknown. The microorganism need not be purified to interact with the second antibody. The microorganism may be killed or inactivated prior to binding to the second antibody to form a complex.

In another aspect, a CD40 agonistic antibody or a portion thereof comprising at least an F(ab) region is provided. The CD40 agonistic antibody or portion thereof is selected from the following: an antibody comprised of SEQ ID NO: 2 and SEQ ID NO: 4: an antibody comprising SEQ ID NO: 14; an antibody or portion thereof comprising a heavy chain variable (V_{H}) region and a light chain variable (V_{L}) region, wherein the heavy chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7 and wherein the light chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and an antibody or portion thereof comprising a heavy chain variable (V_{H}) region and a light chain variable (V_{L}) region, wherein the heavy chain variable region comprises a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22 and wherein the light chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19.

In a further aspect, the CD40 agonistic antibodies may be used to generate a vaccine. In the vaccine, the CD40 agonistic antibody is linked via a linker moiety to an antigen. The antigen may be a peptide. The vaccines may be comprised within an alginate sphere for administration in the food or drinking water.

In a further aspect, methods of enhancing an immune response in a subject are provided. The methods include administering the vaccines or compositions provided herein to the subject in an amount effective to enhance the immune response to the antigen or microorganism. The vaccine or composition may be administered mucosally, may induce both IgG and IgA, in particular sIgA, and induces a rapid response within about 7 days.

In a still further aspect, constructs for production of a vaccine composition. The construct includes a first polynucleotide encoding an anti-CD40 agonistic antibody heavy chain comprising SEQ ID NO: 5, 6, and 7 or SEQ ID NO: 20, 21 and 22 and an anti-CD40 agonistic antibody light chain comprising SEQ ID NO: 8, 9, and 10 or SEQ ID NO: 17, 18 and 19. The polynucleotide is operably connected to a promoter to allow for expression of the anti-CD40 agonistic antibody. The construct may further include a second polynucleotide encoding an antigen and the two polynucleotides may be linked in frame to form a fusion protein when expressed.

In a still further aspect, methods of epitope mapping a polypeptide are provided. Labeled peptides of 8-20 amino acids from the polypeptide are generated and attached to a labeled CD40 antibody via a linker moiety to create a CD40 antibody-peptide complex. The CD40 antibody-peptide complex was administered to a subject and after a period of time that may be as short as 5-7 days sera was collected from the subject and tested for the presence of antibodies able to recognize the polypeptide. Peptides capable of producing antibodies to the polypeptide were identified as antigenic epitopes. These identified antigenic epitopes may be used to develop a vaccine.

### BRIEF DESCRIPTION OF THE DRAWINGS

FIG. 1 is a schematic representation showing the preparation of antibody-peptide complex based on biotin-streptavidin interaction. FIG. 1A shows that biotinylation was limited to the carbohydrate groups on the Fc region of MIg, hence did not interfere with antigen-antibody interaction. FIG. 1B shows that streptavidin (SA) was used for controlled complexing of biotinylated peptide with biotinylated MIg. Mab 2C5 in the 2C5-SA-peptide complex retained its biological function as demonstrated by ELISA.

FIG. 2 is a set of graphs Showing the levels of peptide-specific circulatory IgG (FIG. 2A) and mucosal IRA in trachea (FIG. 2B) elicited by a single s.c. injection of anti-cCD40-guided peptide complex (grey bars, as compared to non-specific MIgG-peptide complex, black bars) as determined by ELISA. Groups of eight five-week old male Leghorn chickens were subcutaneously immunized once with 50 μg Mab 2C5-peptide complex or negative control complex. In each case, error bars represent standard deviations from the mean and the asterisks represent statistical significance (n=8; *P<0.05; **P<0.01; ***P<0.001) compared with non-specific. MIg-peptide complex controls as determined by Student's t-test. At both time points, and for both peptide-specific antibody isotypes (IgG and IgA), a significant immune enhancement caused by CD40 targeting of the peptide cargo to the APCs was observed.

FIG. 3 is a set of graphs showing the levels of peptide-specific circulatory IgG elicited by a single administration of anti-cCD40-guided peptide complex (gray bars, as compared to non-specific MIgG peptide complex, black bars) through oculo-nasal (FIG. 3A), cloacal drinking (FIG. 3B), and oral alginate suspension) (FIG. 3C) routes as determined by ELISA. Groups of eight five-week-old male Leghorn chickens were immunized once with either 50 μg anti-cCD40-guided Mab 2C5-peptide complex or negative control (non-specific) MIgG-peptide complex via three different mucosal routes. Serum and trachea samples were collected 7 and 14 days p.i. and peptide-specific IgG responses were assessed by ELISA. In each case, error bars represent standard deviations from the mean and the asterisks represent statistical significance (n=8; *P<0.05; **P<0.01; ***P<0.001) compared with MIg-peptide complex controls as determined by Student's t-test.

FIG. 4 is a set of graphs showing the levels of peptide-specific mucosal IgA elicited by a single administration of anti-cCD40-guided peptide complex (gray bars, as compared to non-specific peptide complex, black bars) through oculo-nasal (FIG. 4A), cloacal drinking (FIG. 4B), and alginate suspension (oral) (FIG. 4C) mucosal routes as determined by ELISA. Groups of eight five-week-old male Leghorn chickens were immunized once with 50 μg Mab 2C5-peptide complex or negative control complex via various mucosal routes and serum and trachea samples were collected from chickens at 7 and 14 days p.i. In each case, error bars represent standard deviations from the mean and the asterisks represent statistical significance (n=8; *P<0.05; **P<0.01; ***P<0.001) compared with MIg-peptide complex controls as determined by Student's t-test.

FIG. 5 is a set of graphs showing the net elect of 2C5-peptide complex on induced circulatory IgG (FIG. 5A) and mucosal sIgA (FIG. 5B) immune response through various mucosal and classic s.c. routes at 7 and 14 days post administration. The CD40 targeting induced net effect was calculated as [Average (S/P) value of treatment from each route]−[Average (S/P) value of corresponding MIg control].

FIG. 6 is a schematic depiction of one embodiment of the invention showing the molecular structure of a bispecific antibody complex consisting of a scaffold or linker protein molecule (biotin-streptavidin), two agonistic chicken anti-CD40 antibody molecules and two antibodies specific for M2e (a conserved antigen on Influenza).

FIG. 7 is a schematic depiction showing how the bispecific antibody complex of FIG. 6 acting as an adjuvant can be complexed with a microorganism such as a virus (Influenza) even from a crude source of the virus such as allantoic fluid or a cellular lysate. The adjuvant composition is simply incubated with a crude preparation of the microorganism to form the complex

FIG. 8 is a schematic depiction showing how the adjuvated virus of FIG. 7 can interact with an antigen presenting cell to target CD40 and enhance the immune response of the subject to the virus. The antigen-presenting cells of the host express CD40 and the CD40 antibody targets the complex to the antigen presenting cells and induces signaling via CD40 to enhance both the cell mediated and humoral immune response.

FIG. 9 is a graph showing the results of an ELISA against cCD40 and CD205 demonstrating the scFv anti-CD40 resulting from the panning procedure recognizes cCD40, but an antibody targeting CD205 did not recognize the cCD40.

FIG. 10 is a graph showing the results of an ELISA against cCD40 of the purified scFv anti-cCD40 DAG 1.

FIG. 11 is a set of photographs showing that the anti-cCD40 DAG1 recognized CD40 on the surface of chicken B cells (DT40; FIG. 11A) and macrophages (HD11, FIG. 11B) by immunocytochemistry.

FIG. 12 is a photograph showing in vitro agglutination of DT40 B cells by the scFv anti-cCD40 DAG1.

FIG. 13 is a graph showing that purified anti-cCD40 scFv (DAG1) is agonistic for cCD40 and stimulates production of nitric oxide by HD11 macrophages.

FIG. 14 is a graph showing the survival post-challenge of chickens after vaccination with the indicated material. CD40 agonistic antibody complexed with the three M2e antibodies were able to increase survival after challenge equal to a commercial vaccine.

FIG. 15 is a graph showing the ability of sera from chickens vaccinated with the indicated vaccines one week earlier to inhibit Influenza-mediated hemagglutination.

FIG. 16 is a graph showing the hemagglutination assay results for three different clones of anti-M2e showing each individual bird's results.

FIG. 17 is a set of graphs showing the mean hemagglutination value for the various groups. FIG. 17A shows the mean value when all dilutions are combined and clone C was significantly better than the controls or other clones. FIG. 17B shows the comparison with all the controls separated the Group C complex was not significantly better than the commercial vaccine or the killed virus, but was numerically better than either.

FIG. 18 is a graph showing the ratio of antibodies produced seven days after immunization with the indicated peptide-CD40 agonistic antibody complexes as compared to the day of immunization.

### DETAILED DESCRIPTION

In chickens, as in mammals, most infectious diseases begin at the mucosal surface of the respiratory or the digestive tract. Local immunity is hence crucial in host defense against pathogens that invade and colonize these surfaces. Mucosal immunization (as opposed to injection under the skin or in the muscle) with the vaccine, especially if it is nota live vaccine, can lead to enhanced mucosal immune responses but is hampered by the limited absorption of the vaccine through the mucous membranes. Mucus that covers the surface of so-called Mucosa-Associated Lymphoid Tissue (MALT) often prevents attachment and uptake of vaccines by immune cells. In addition, when administered orally, the bird's crop and gizzard (or a mammal's stomach) can also break down the vaccine mechanically or enzymatically before it reaches the intestinal immune tissue. Even if the vaccine reaches the MALT in a fashion that can be recognized by the local immune system, not all vaccines stimulate the Antigen-Presenting Cells (APCs; the “sentinel cells” of the immune system) equally well. Thus, repeated large doses (20-100 μg/dose) of a vaccine are often required for an effective sIgA response. Using the technology disclosed here, a single immunization with an antibody-guided vaccine complex targeting the CD40 receptor molecule (which is expressed on chicken APCs) resulted in significant vaccine-specific systemic IgG and mucosal sIgA responses as early as 1 week post-vaccination. All the administration routes that were tested in the Examples (mucosal, including oral, eye drops and cloacal, but also subcutaneous application) resulted in comparable IgA responses, and a very small amount of the vaccine was sufficient to elicit significant (P<0.001) vaccine-specific mucosal IgA responses. After a single sub-cutaneous injection, the anti-CD40 antibody-peptide complex induced significant systemic IgG responses on day 7 and 14 post-infection. Compared to conventional adjuvants, the anti-cCD40 monoclonal antibody-peptide complex is able to mimic the biological role of CD4⁺ T cells by targeting APCs, including B-cells, and further enhancing CD40 downstream signaling and subsequent immunoglobulin class-switching from IgM to IgG or IgA.

Interestingly, a single sub-cutaneous injection with the CD40 monoclonal antibody-peptide complex also induced a significant mucosa/ peptide-specific sIgA immune response as early as 7 days post infection as measured by ELISA in mucosal extracts from trachea segments. In the past, the most effective strategy to induce both systemic and mucosal immunity was by using a combination of priming and boosting through the mucosal and systemic routes, respectively.

To the best of our knowledge, past literature states that parenteral immunization alone is unable to prime the specific mucosal immune response in mammals because circulatory resting B-cells in the periphery express different homing receptors compared to the mucosal B-cells in the common mucosal immune system (CMIS) (Macpherson et al., 2008, Mucosal Immunol 1:11-22; Mei et al., 2009, Blood 113: 2461-2469; Mestecky, 1987, J Clinical Immunol 7:265-276; Neutra and Kozlowski, 2006 Nat. Rev. Immunol. 6, 148-158). However, this concept has recently been challenged, and a system similar to the CMIS has been proposed to explain that parenteral immunization might also contribute to antibody-mediated mucosal immunity in humans (Fernandes, 2012, Correlates of mucosal Immoral immunity in peripheral blood, In: Medical Sciences, Vol. PhD. McMaster University, McMaster University Libraries Institutional Repository, page 163). Recently, activated B-cells were shown to express the mucosal homing receptor, chemoattractant cytokine receptor 10 (CCR10). CCR10⁺ B-cells in circulation are considered to be in transit between a systemic (peripheral) lymphoid tissue and mucosal effector tissues, where they are transformed into polymeric IgA-secreting plasma cells (Fernandes and Snider, 2010, Int-immonol, 22, 527-540). Polyclonal anti-CD40 antibodies have been reported to initiate the CCR10 expression on recently activated memory B-cells in mice in vitro (Bernasconi et al., 2002; Science 298, 2199-2202). On the other hand, CCR10 ligand is expressed in all mucosal effector sites (Mora and von Andrian, 2008; Mucosal Immunol. 1, 96-109). In mammals, polyclonal anti-CD40 antibodies were also reported to mediate the expression of CXCR4 on IgG-secreting B cells. CXCR4 is a homing receptor for homing of B-cells to the bone marrow and to secondary lymphoid organs. Without being limited by theory, we believe this provides a plausible mechanistic explanation for why parenteral immunization with an anti-CD40 monoclonal antibody-peptide complex may indeed be capable of inducing both significant peptide-specific systemic IgG and mucosal sIgA immune responses.

Taken together, these results made it plausible to test whether a single parenteral or mucosal immunization with a cCD40 monoclonal antibody guided antigen complex can induce not only a fast and long-lived systemic IgG immune response, but also a rapid local mucosal sIgA response. Therefore, this new platform may have the potential to be widely used for immunization of chickens and other animals through mucosal and: or parenteral administration in cases where both systemic and mucosal immunity are highly desirable. The latter is especially important for vaccination of poultry, in which most pathogens invade through the mucosal surfaces of the respiratory or digestive tract. Even though there are unresolved questions about the mechanism and the micro-environment of the interaction of APCs and cCD40-peptide complex, the results obtained in the current study are encouraging, and there seems to be considerable potential for the development of safe, effective and affordable vaccines.

The main advantages of this approach are: (1) fast immune reponses; (2) production of IgA, the only antibody class that is protective on mucosal surfaces; (3) single administration regimen; (4) easy and inexpensive routes of administration; (5) lesion-free injection sites thanks to its formulation in a physiological buffer; and (6) long-lived immunological memory. In addition, in one embodiment we have produced the antibody portion of this vaccine by genetic engineering methods that permit attachment of this “guiding antibody” to any protein antigen of interest and production of a single fusion protein in a production platform that is capable of low cost, scalable production of large quantities of the vaccine and ease of transition to new systems or emerging infectious diseases. This vaccine has been characterized in tissue culture (“in vitro”) and will be produced in the green alga Chlamydomonas reinhardtii, to be tested in live animal: as described in the Examples. The vaccine will also be tested without prior extraction and purification from the algae to enable us to produce it at even lower cost. We expect this configuration of the vaccine to work similarly to the alginate used in the Examples for oral administration.

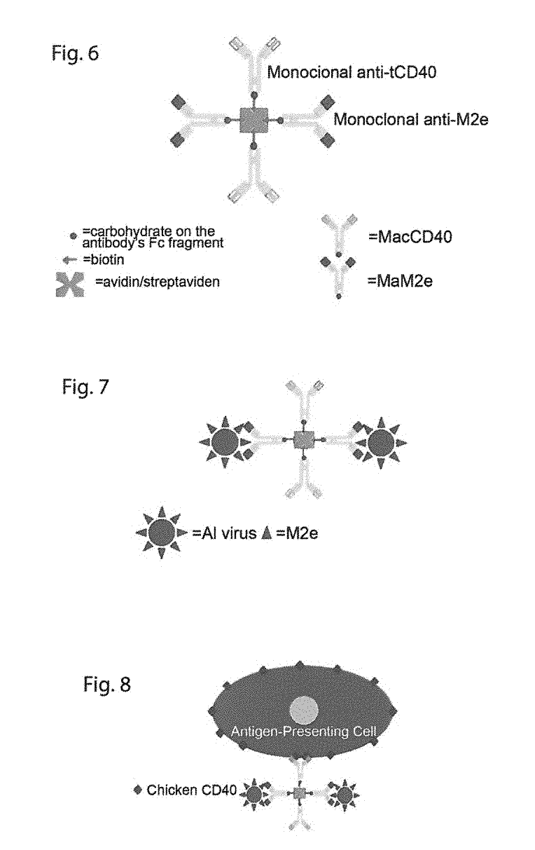

In another embodiment of the invention shown in FIGS. 6-8, CD40 antibodies are complexed with antibodies capable of specifically binding to a microorganism. This approach allows formation of an adjuvant-immunogen complex with minimal information about the microrganism. For example, the serotype of a virus or bacterial strain need not be known as long as the antibody is capable of binding to an invariant protein motif (“epitope”) on the surface of the microorganism. Influenza viruses and Salmonella have a wide variety of proteins on their surface that are highly variant and related to the virulence of the organism, but the antibody for use in the current methods may be selected to bind an invariant or not as highly variant protein motif on the surface of the microorganism such that a simple binding assay may be used to complex inactivated microorganisms to the CD40 complex adjuvant composition for use as a vaccine. This approach avoids using any recombinant technology and thus may be more acceptable in countries or locales adverse to recombinant DNA technology. In addition, this technology can be rapidly developed in response to an outbreak with a new variety (i.e. distinct serotype or in influenza a distinct HN profile) of the microorganism and can be used without any need to isolate the microorganism prior to binding to the CD40 antibody complex. The production of vaccines including the CD40 antibody complexed with an antibody specific for the micoorganism and the inactivated microorganism may be made without the need for clean rooms or other technology and could even be generated in the field. The complex will be targeted to antigen-presenting cells in the host and the agonistic CD40 antibody will help induce both humoral and cell-mediated immunity against the microorganism.

Production of antibody-guided CD40 targeted mucosal vaccines using the above principle is feasible against nearly all pathogens even newly arising pathogens because there is no need to identify the target antigens precisely prior to or in conjunction with vaccine development. Production of vaccines in which a suitable target (proteinaceous or other) has been identified can also be streamlined. These vaccines may be used not only in chickens but also in other meat producing animals, ranging from fish to mammals, as long as the CD40 guiding antibody is directed against the host-specific CD40 molecule. Agonistic CD40 antibodies have been identified in several other animals including human, mouse, rat, pig, dog, horse, cows, pigs, goats, sheep, as well as chickens disclosed herein. Several CD40 sequences are provided as SEQ ID NOs: 54-56 and antibodies can be raised against the specific CD40 for each species. Many of these CD40 antibodies and specifically CD agonisitic antibodies are commercially available. See Linscott's Directory of immunological and Biological Reagents.

One of the chicken CD40 agonistic antibody used herein is a mouse antibody but those of skill in the art will appreciate that the Fc portion of the antibody can be altered to make the antibody more compatible with the system in which it is used. Thus the antibody provided herein as SEQ ID NO: 2 (heavy chain) and SEQ ID NO: 4 (light chain) referred to in the Examples as 2C5 or SEQ ID NO: 14 (single chain variable fragment (scFv)) referred to in the Examples as DAG-1, may be made in a “chickenized” form such that the Fe portion and the non-CDR regions may be replaced with homologous host-compatible antibody backbone sequences to minimize the immune response to the antibody backbone itself. In addition, the antibodies may be made either recombinantly or via enzyme digestion (i.e. papain or pepsin) into smaller portions of the antibodies and include only the F(ab) portion of the antibody, such as an R(ab)₂ fragment. The CDR regions for both chicken CD40 antibodies used in the Examples have been identified. For the antibody designated as 2C5 and provided in SEQ ID NO: 2 and SEQ ID NO: 4, the heavy chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7 and the light chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10. For the antibody designated as DAG-1 and provided in SEQ ID NO: 14, the heavy chain variable region comprises a CDR I comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22 and wherein the light chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19. Those of skill in the art may use methods available to make the antibody more compatible for use and activity in chickens or to generate any of the antibody variants known to those of skill in the art, including but not limited to bispecific antibodies, diabodies, linear antibodies, nanobodies, Fab, Fab′, F(ab)₂, Fv or scFv. Thus the methods and compositions described herein include the antibodies or portions thereof which are antigen-binding fragments of the antibodies. Suitably the portions of the antibodies include the indicated CDR regions and maintain the affinity for their target, CD40, and also maintain the ability to ligate the CD40 receptor subunits (which is required for the agonistic bioactivity) and induce CD40 signaling when bound to CD40 on an antigen-presenting cell.

Similarly antibodies directed to CD40 of other animals can begenerated and used in the methods and compositions described herein. For example anti-CD40 antibodies directed to turkey, bovine, porcine, goats, sheep, fish, dogs, cats, or other domesticated animals can be generated and used in the methods and compositions described herein. See SEQ ID NO: 54-56. These antibodies can be made in animals such as mice or rabbits and then modified to make them more compatible for use in the methods in the animal for which they are specific, i.e., the antibodies can have the constant regions swapped out for those of the target animal.

Alternatively phage display or other recombinant systems may be used to generate CD40 antibodies. In addition, CD40 antibodies and agonisitic CD40 antibodies are commercially available for several species, in particular mouse and human. An antibody is agonistic for CD40 if it is capable of inducing signaling within the target cell expressing CD40. The signalling via CD40 results in increased expression of CD 40 and TNF receptors on the surface of the antigen-presenting cells and induces production of reactive oxygen species and nitric oxide, and B cell activation leading; to isotype switching. Thus the inventors believe the agonistic effects of the CD40 antibody are at least partially responsible for the large amount of IgG and IgA produced very quickly after immunization with the CD40 antibody complexes described herein. The CD40 antibodies provided herein may be made from hybridoma cells, purified from ascites fluid or from cells genetically engineered to express the antibody. Those of skill in the art will appreciate that there are a wide variety of ways available to generate an antibody. The antibody can be linked with a linker moiety directly to an antigen or may be linked to a second antibody capable of specifically binding to a microrganism, such as a virus, bacterium, yeast, or single celled parasite or protist. The microorganism may be inactivated or killed by any means known to those of skill in the art but would include heat killing, paraformaldehyde killing, use of antibiotics or alcohol. The linker can be a peptide linker (i.e. in a fusion protein) to link a peptide antigen to an antibody or a may be a non-peptide covalent or non-covalent bond or other chemical linker or may rely on a receptor-ligand interaction. In the Examples, the antibodies are labeled with biotin and streptavidin is used as the linker moiety. An N-hydroxysuccinimide linker or a thioester linker may be used. Other means of linking the antibodies to an antigen, pathogen or part thereof are available.

The CD40 agonisitic antibodies are used in adjuvant compositions and vaccines as described in the examples and appended claims. In one embodiment, an adjuvant composition comprising at least one first CD40 agonistic antibody or portion thereof comprising at least two Fab regions capable of specifically binding CD40 and inducing CD40 signaling, at least one second antibody or portion thereof comprising at least two Fab regions capable of specifically binding a microorganism, at least one label attached to the at least one first CD40 agonistic antibody or portion thereof, at least one label attached to the at least one second antibody or portion thereof, and a linker moiety capable of specifically binding to the labels attached to the antibodies. The first CD40 agonistic antibody and the second antibody are bound to the linker moiety to form a complex, which is also referred to as the CD40 antibody-second antibody complex.

The second antibody in some of the adjuvants described herein is an antibody capable of specifically binding to a microorganism. The antibody may bind specifically to an antigen or epitope present on the surface of the microorganism. The microorganism may be a virus, bacteria, yeast, or protists. The microorganism may be a pathogen, such as Influenza or a bacterial pathogen or a vaccine vector such as a bacterial or viral vaccine vector. The bacterial pathogen may be a pathogen prone to genetic variation or prone to generate escape variations when under selective pressure and the antibody could be directed to a conserved epitope to allow for autologous pathogen fragments to be combined with the CD40 antibody to provide rapid vaccination in response to an emergent pathogen. The serotype of the microorganism need not be known if the antibody binds specifically to another epitope available on the surface of the microorganism. For example, the second antibody may be specific for a pan-expressed antigen such as M2e for Influenza and the antibody would bind to M2e expressed on the surface of inactivated Influenza virus particles in an Influenza virus vaccine to adjuvate the Influenza vaccine by combination with the CD40 antibody. Other bacteria or viruses for which the second antibody may be specific include but are not limited to influenza virus, Salmonella, Clostridium, Campylobacter, Escherichia, Shigella, Helicobacter, Vibrio, Plesiomonas, Edwardia, Klebsiella, Staphylococcus, Streptococcus, Aeromonas, Foot and Mouth virus, porcine epidemic diarrhea virus (PEDv), and Porcine reproductive and respiratory syndrome virus (PRRSV). For example, the antigens or bacterial vaccine vectors identified in U.S. Pat. No. 8,604,198, International Publication Nos. WO2009/059018, WO2009/059298, WO2011/091255, WO2011/156619, WO2014070709, WO 2014/127185 or WO 2014/152508. Several peptides to which the second antibody may bind specifically include, but are not limited to those in SEQ ID NO: 25-53 or 57-58, SEQ ID NO: 58 was the target for the second antibody used in the Examples.

The adjuvants comprising CD40 antibody provided herein may be used as vaccines or as an adjuvant for use in combination with known vaccines. Combination of the adjuvants described herein with a known vaccine can substitute for another adjuvant or be used in conjunction with an established vaccine to increase the systemic immune response, increase the rapidity of the development of the immune response or allow for production of a mucosal immune response to the vaccine. Vaccines may also be made by combining the adjuvant composition (including the CD40 antibody-second antibody complex) by binding the second antibody to a microorganism to produce a novel vaccine. These novel, non-recombinant vaccines can be made quickly after the cause of an infectious outbreak is identified and do not require that the causative agent is characterized or isolated to produce an effective vaccine. The vaccines are inexpensive to produce and can be made from sources of the infectious agent (microorganism) such as allantoic fluid with little or no purification of the microorganism. The microorganism may be Influenza virus, any of the microorganisms specifically recited herein or any other microorganism for which a vaccine is needed. For oral administration the vaccine including the CD40 adjuvants described herein may be included in a protective coating such as alginate spheres. The adjuvants may also be produced using the genetic engineering constructs provided herein such that the vaccine is produced by the cells and may be fed to the subject. For example, cells of a plant, yeast or alga could be genetically engineered to produce an edible vaccine, capable of surviving in the gastrointentinal tract of the subject.

In an alternative embodiment, the CD40 antibody is linked to an antigen by a linker moiety such as the Clostridium perfringens α-toxin used in the Examples. See SEQ ID NOs: 59-83. Any other antigens known to stimulate an immune response may be used similarly. The antigen may be linked via a peptide linkage to form a fusion protein between the antibody and the antigen or may be chemically linked either covalently or non-covalently through a linker moiety as described above.

The adjuvants and vaccines described herein may be used to make pharmaceutical compositions. Pharmaceutical compositions comprising the adjuvants and vaccines described above and a pharmaceutically acceptable carrier are provided. A pharmaceutically acceptable carrier is any carrier suitable for in vivo administration. Examples of pharmaceutically acceptable carriers suitable for use in the composition include, but are not limited to, water, buffered solutions, glucose solutions, oil-based or bacterial culture fluids. Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, and dextran), proteins such as albumin or casein, protein-containing agents such as bovine serum or skimmed milk and buffers (e.g., phosphate buffer). Especially when such stabilizers are added to the compositions, the composition is suitable for freeze-drying or spray-drying. The composition may also be emulsified.

The adjuvants and vaccines may be administered in combination with other vaccines in any order, at the same time or as part of a unitary composition. The compositions may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more.

Treating a subject as used herein refers to any type of treatment that imparts a benefit to a subject afflicted with a disease or at risk of developing the disease, including improvement in the condition of the subject (e.g., in one or more symptoms), reduction in mortality, reduction in morbidity including weight loss or feed conversion rate, delay in the progression of the disease, delay the onset of symptoms or limiting the severity of symptoms, etc. The treatment may be due to an increase or enhancement of the immune response to an organism in the subject. The immune response in response to administration of the vaccine or adjuvant may be an increased humoral or cell-mediated immune response directed to the target antigen or microorganism.

Methods of enhancing immune responses in a subject by administering to the subject the vaccines described herein in an effective amount to enhance the immune response to the antigen are provided. The immune response that is enhanced may include a T cell or B cell response. Suitably the enhanced immune response allows class switching such that IgG and sIgA directed to the antigen, microorganism or vaccine vector is generated. A single dose of the vaccine can induce a robust immune response within a short period of time. Suitably an enhanced immune response is measurable after seven days. In particular a strong IgA response can be generated in this short time span.

An effective amount or a therapeutically effective amount as used herein means the amount of the adjuvant or vaccine that, when administered to a subject for treating a state, disorder or condition is sufficient to elect a treatment (such as an enhanced immune response). The effective amount will vary depending on the exact composition and its formulation, the disease or pathogen being targeted by the vaccine and its severity and the age, weight, physical condition and responsiveness of the subject to be treated.

The compositions described herein may be administered by any means known to those skilled in the art, including, but not limited to, mucosal, oral, topical, intranasal, intraperitoneal, parenteral, intravenous, intramuscular, subcutaneous, intrathecal, transcutaneous, nasopharyngeal, cloacal, ocular, or transmucosal absorption. Thus the compositions may be formulated as an ingestible, injectable, topical or suppository formulation. Administration via the mucosal route includes oral via the drinking water, via spraying the birds, or via inclusion in or on the feed. Also included are cloacal, nasal, or oral gavage. The compositions may also be delivered with in a liposomat or time-release vehicle or encased within alginate spheres. Administration of the compositions to a subject in accordance with the invention appears to exhibit beneficial effects in a dose-dependent manner. Thus, within broad limits, administration of larger quantities of the compositions is expected to achieve increased immune responsiveness up to an optimal dose. In general once an optimal dose is achieved further increases in administration produce no advantage in terms of response. Moreover, efficacy is also contemplated at dosages below the level at which toxicity or adverse responses are seen.

It will be appreciated that the specific dosage administered in any given case will be adjusted in accordance with the compositions being administered, the condition of the subject, and other relevant medical factors that may modify the activity of the compositions or the response of the subject, as is well known by those skilled in the art. For example, the specific dose for a particular subject depends on age, body weight, general state of health, diet, the timing and mode of administration, the rate of excretion, and medicaments used in combination. Dosages for a given patient can be determined using conventional considerations, e.g., by customary comparison of the differential activities of the compositions of the invention and of a known agent such as a vaccine not combined with the anti-CD40 based adjuvant described herein, such as by means of an appropriate conventional pharmacological or prophylactic protocol.

The maximal dosage for a subject is the highest dosage that does not cause undesirable or intolerable side effects. The number of variables in regard to an individual regimen is large, and a considerable range of doses is expected. The route of administration will also impact the dosage requirements. It is specifically contemplated that pharmaceutical preparations and compositions may palliate or alleviate symptoms of the disease, i.e. lead to reduced severity if exposed to the pathogen or reduced morbidity or mortality after exposure or may prevent the subject from contracting a disease after subsequent exposure to the pathogen for which the vaccine or antigen was specific.

Suitable effective dosage amounts for administering the compositions may be determined by those of skill in the art, but typically range from about 1 microgram to about 1,000 micrograms per kilogram of body weight or per dose, although they are typically about 10 to 100 micrograms or less per kilogram of body weight or per dose. In general, a single dose is administered and is effective to induce an immune response. In some cases the initial dose is followed by a boost, which may be with the same or a distinct composition provided at least two weeks after the first administration. The boost may be administered 2-6, 2-4, or optionally 2-3 weeks after the initial dose.

Although the consequence of phylogenetic separation of chickens from the reptile ancestor of mammals was about 300 million years ago, chickens are also endowed with a sophisticated mucosal immune system including a series of redundant protective mechanisms. Chickens lack encapsulated lymph nodes such as are found in mammals, but rather possess diffuse lymphoid tissues. Chickens were used as a model system in the Examples, but the methods used in chickens are expected to elicit similar immune responses in mammals and in particular in other domesticated animals and humans. Mucosal immune responses are most efficiently induced when the antigen is delivered directly onto mucosal sites through mucosal routes. Mucosal immune sites are interconnected by a common mucosal immune system (CMIS) whereby stimulation of an inductive site (where the immune response initiated), the resulting immune response to be disseminated to the distal effector sites of the mucosa.

Constructs for production of a vaccine composition comprising a first polynucleotide encoding an anti-CD40 agonistic antibody operably connected to a promoter to allow for expression of the anti-CD40 agonistic antibody are also provided herein. The anti-CD40 antibody comprises a heavy chain which includes CDR1 (SEQ ID NO: 5 or 20), CDR2 (SEQ ID NO: 6 or 21) and CDR3 (SEQ ID NO: 7 or 22) and a light chain which includes CDR1 (SEQ ID NO: 8 or 17), CDR 2 (SEQ ID NO: 9 or 18) and CDR3 (SEQ ID NO: 10 or 19). The remaining portions of the antibody may be those of SEQ ID NO: 2 and SEQ ID NO: 4 or may be engineered to be more compatible with the host, i.e. the chicken, such that administration of the adjuvants and vaccines does not elicit an immune response targeted against the mouse portions of the antibody. Alternatively other constructs can be made such as a single chain variable fragment (scFv) as shown in SEQ ID NO: 14. Methods of engineering antibodies are available to those of skill in the art and include other antigen-binding derivatives of the antibodies described herein based on the CDR regions provided above, including but not limited to, scFVs, single domain antibodies, nanobodies, chimeric antigen receptors, diabodies and other bi- or multi-specific antibodies.

The antibody may be further engineered to make the construct more useful. The promoter may be a constitutive promoter or an inducible promoter to generate large amounts of antibody within a small time frame. The first polynucleotide may be engineered to contain a secretory signal such that the polypeptide encoded by the polynucleotide is secreted from the cells. The first polynucleotide may be labeled with a detectable label or a label that makes isolation or purification of the polypeptide straightforward. Labels include fluorescent labels, or protein tags such as a His tag. See SEQ ID NO: 23-24. The construct may contain a multi-cloning site to make further genetic engineering or addition of a second polynucleotide encoding an antigen straightforward. The second polynucleotide may be linked in frame with the first polynucleotide to generate a fusion protein containing both the CD40 antibody and the antigen. As noted above, antigens for incorporation in the construct include but are not limited to those disclosed in U.S. Pat. No. 8,604,198, International Publication Nos. WO2009/059018, WO2009/059298, WO2011/091255, WO2011/156619, WO2014070709, WO2014/127185 or WO2014/152508 and those provided in SEQ ID NO: 25-53 and 57-83. Cells comprising the constructs are also provided. The cells may be bacterial, yeast, algal, plant or mammalian cells capable of expressing the polynucleotides generating the polypeptides and compositions described herein.

Methods of epitope mapping are also provided herein. The methods provided herein allow rapid identification of potential linear B cell epitopes within a polypeptide/protein of interest and can be applied to any proteinaceous target. The methods rely on linkage of peptides of 8-20 amino acids from the polypeptide to a CD40 antibody. Suitably the peptides are made synthetically and linked via a linker moiety to the CD40 antibody to create a CD40 antibody-peptide complex. This step avoids the need for any recombinant biology to generate the antigens. Synthetic peptides may be prepared using methods known to those of skill in the art and may be made by commercial vendors. The synthetic peptides may be labeled to provide a simple means of complexing the peptides to the CD40 antibody. For example the CD40 antibody and the peptide may be biotinylated and then streptavidin or avidin may be used to link the CD40 antibody to the peptides. Other means of attaching peptides to a CD40 antibody via a linker moiety are provided above. The peptides may be generated such that they span an entire polypeptide or may be selected to focus on areas within the polypeptide that are likely to contain a B cell epitope. See Example and SEQ ID NOs:59-83. These peptides are generally soluble in water and polar. Computer programs for predicting B cell epitopes in polypeptides are available and may be used in conjunction with the methods described herein.

The CD40 antibody-peptide complex once generated is then administered to a subject and after a period of time that may be as short as 5-7 days, sera are collected from the subject and tested for the presence of antibodies able to recognize the full-length native polypeptide or portions thereof. Peptides capable of producing antibodies to the polypeptide are identified as antigenic epitopes. The sera may be tested using any method available to those of skill in the art, including, but not limited to ELISA assay, Western blot, immunofluorescence, FACS analysis or a functional protein assay. Functional protein assays include neutralization or agonist assays. A neutralization assay tests for the ability of the sera to block function of the native protein. An agonist assay tests for the ability of the antibodies in the sera to bind to and activate the protein's function. The sera and antibodies capable of binding or otherwise performing in the assays are indicative of antigenic epitopes. These identified antigenic epitopes may be used to develop a vaccine or to develop an antibody specific for the polypeptide as a whole. A protein can be epitope mapped using this technique in a few weeks and this can be done in a test subject rather than in mice. For example, chickens may be used as the subject. Traditionally this process has taken more than one month and repeated boosts to generate a robust immune response for In vitro testing.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise dearly contradicted by context. The use of any and all examples, or exemplary language provided herein, is intended merely to facilitate the disclosure and does not necessarily imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements arc also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meat t as limitations on the scope of the invention or of the appended claims

### EXAMPLES

### Example 1

### Generation and Use of Chicken CD40 Antibodies to Induce IgA to Peptides

### Materials and Methods

Anti-cCD40 Monoclonal antibody (Designated as 2C5)

Our lab has previously reported the development of an agonistic anti-cCD40 Mab, designated as 2C5 (Chen et al., 2010b Development and Comparative Immunology 34: 1139-1143). Mab 2C5 was made against the recombinant extracellular domain of cCD40 (cCD40_{ED}), (recombinant cCD40 obtained from CVM-VTPB). This Mab recognized and bond to CD40 as expressed on primary chicken B-cells and macrophages, DT40 B-cells, and HD11 macrophages, Mab 2C5 also induced NO production in HD11 macrophages, and stimulated DT40 B-cell proliferation (Chen et al., 2010b). These results demonstrated that 2C5 induces downstream CD40 signaling after binding to CD40 and is thus agonistic. Mab 2C5 mimicked at the very least partially the functions of the chicken's natural CD40 ligand, CD154. Chen et al. (2012, Immunol Methods 378: 116-120) also reported that targeting an antigen to chicken CD40⁺ APCs can significantly enhance antigen-specific circulatory IgG responses and thus induce fast immunoglobulin isotype-switching (Chen el al., 2012).

### Streptavidin-Mediated Complexing of Peptide to Mouse Antibody

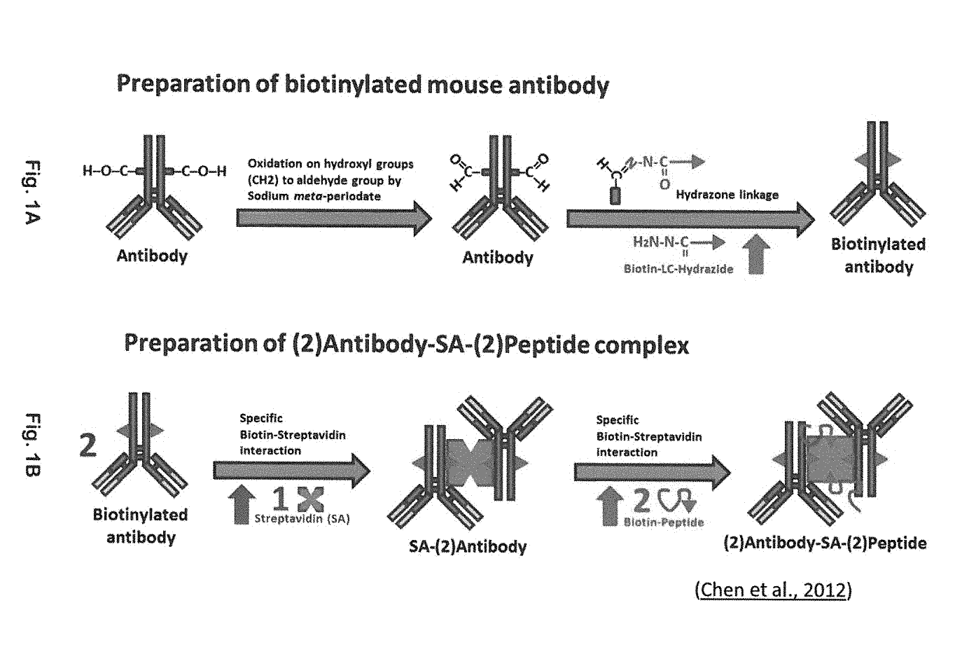

The anti-CD40 Mab-peptide complex (designated as “Mab 2C5-peptide complex”) and control complexes (where non-specific MIgG was substituted for anti-cCD40 Mab 2C5) were prepared essentially as described previously (Chen et al., 2012). Briefly, anti-chicken CD40 Mab 2C5 (SEQ ID NO: 2 and 4) and non-specific control mouse immunoglobulin (MIg) were directionally biotinylated by derivatization of the carbohydrate moieties on the Fc fragment. Biotinylation and retention of cCD40-binding capacity were verified by enzyme-linked immunosorbent assay (ELISA; results not shown). A synthetic amino-terminally biotinylated peptide (b-NAWSKEYARGFAKTGK; SEQ ID NO: 57) and streptavidin (SA) were used in a stoichiometrically controlled complexing reaction of the biotinylated peptide with biotinylated 2C5 (or MIg) in a ratio of 1 SA molecule to 2 peptide molecules and 2 immunoglobulin molecules (FIG. 1).

However, because an immunoglobulin-peptide complex is likely susceptible to the enzymatic and acidic pH environment of the gastrointestinal tract, protective encapsulation of the immunoglobulin-peptide complex in an alginate matrix was considered a logical precaution when oral administration was required. Alginate encapsulation is a viable approach for oral delivery of antigens, and the entrapped functional immunoglobulin-peptide complex in fine alginate spheres can be safely delivered to the appropriate site, (such as the Peyer's patches), despite the harsh gastrointestinal environment that would likely degrade any non-protected protein (Desai and Schwendeman, 2013, J of Controlled Release 165: 62-74). For this study, encapsulation of Mab 2C5-peptide complex and MIg-peptide complex in alginate spheres was performed essentially as reported by Park and colleagues (Bowersock et al., 1999, Vaccine 17:1804-1811) with minor modifications. To prepare Mab 2C5-peptide or non-specific MIg-peptide complex in the form of alginate-protected particles, the molecular complex was freshly produced and then gently mixed with 3% (w v) sodium alginate (Sigma-Aldrich, St Louis, Mo.) in phosphate buffered saline (PBS), pH 7.4, to obtain a homogeneous solution. The resulting solution was then extruded drop-wise through a 23-gauge needle attached to a 1 mL plastic syringe into 3% (w/v) CaCl₂ solution with gentle stirring for 30 minutes at room temperature. Gelified alginate spheres were separated from the CaCl₂ solution by centrifugation at 3,000 g for 10 minutes at 4° C. and were further washed three times with PBS, pH 7.4. To reduce the porosity of the alginate spheres, they were stabilized by coating them in 0.3% (w/v) poly-L-lysine solution with gentle stirring for 30 minutes at room temperature. Poly-L-lysine coated alginate spheres were then washed three times with PBS, pH 7.4. These alginate spheres could be stored at 4° C. until use. On the day of use, the alginate spheres were mechanically fragmented using an IKA® T10 basic ultra turrax homogenizer (Sigma-Aldrich) to form a suspension of smaller microspheres prior to oral administration of the suspension. The morphological characteristics of the alginate spheres were microscopically verified using a hemocytometer. The mean size of the alginate spheres prior to fragmentation was around 1.5 mm in diameter, and the diameter of (fragmented) alginate microspheres in suspension ranged from 10 to 100 μm.

Immunization of Chickens with Mab 2C5-Peptide Complex in Solution or as Alginate-Encapsulated Mab 2C5-Peptide Complex Microsphere Suspension

Four-week old male Leghorns were randomly assigned to different groups (n=16/group). Non-encapsulated Mab 2C5-peptide complex (or “blind”, non-specific MIg-peptide complex, used as negative control) solution in PBS (pH=7.4), was used for immunization via subcutaneous (s.c.) injection, via cloacal drinking (bursal route), and via intraocular drop (oculo-nasal route) administration. For s.c. injection, 50 μg Mab 2C5-peptide MIg-peptide complex in a volume of 0.5 mL emulsified PBS (containing 5% (v/v) squalene and 0.4% (v/v) Tween 80 (Sigma-Aldrich), pH=7.4) was injected in the nape of the neck of each chicken. For cloacal drinking, 50 μg Mab 2C5-peptide MIg-peptide complex in a volume of 150 μL PBS was administrated by dropping the immunogen solution onto the cloacal lips of chickens using a P200 pipette. For intraocular immunization, 50 μg 2C5-peptide/MIg-peptide complex in a volume of 40 μL PBS was administered as eye drops in both eyes of the chickens. For oral immunization with alginate sphere suspension, the immunogen was gently dropped into the oral cavity of the restrained chickens until they spontaneously swallowed it Alginate suspension containing 50 μg 2C5-peptide complex in a volume of 2 mL PBS, pH 7.4, using a pasteur pipette was administered to each of the 16 chickens. Chickens that received the immunogen through cloacal or oral administration were fasted 24 hours prior to immunization to prevent the immunogen from being regurgitated or expelled. The conditions for animal use in this study were approved by the Institutional Animal Care and Use Committee of Texas A&M University, in accordance with the guidelines of the American Association for Laboratory Animal Science.

### Quantification of Peptide-Specfic Serum IgG in by ELISA

Levels of peptide-specific IgG in circulation were determined by ELISA essentially as described previously (Chen el al., 2012). Briefly, biotinylated-peptide was first complexed with goat anti-biotin antibody (Thermo Scientific) on a rotator at 37° C. for one hour in equimolar ratios. Next, the peptide-goat antibody complex (5 μg/mL) was coated overnight on flat-bottom, 96-well microliter plates (Thermo Scientific) in 0.05M carbonate-bicarbonate buffer, pH 9.6. at 4° C. Excess unadsorbed peptide-goat antibody complex was removed by rinsing the plates, and then they were blocked with PBS containing 5% (w v) bovine serum albumin (BSA) (Rockland Immnunochemicals Inc., Gilbertsville, Pa.) for one hour at 37° C. Peptide coated wells were washed with PBS containing 0.2% (v/v) Tween 20 (SIGMA) (PBST) and then incubated with chicken serum samples diluted (1:100) in PBST containing 3% (w/v) BSA overnight at 4° C. The plates were then washed as described above and incubated with horseradish peroxidase-conjugated rabbit anti-chicken IgY (H+L) (Thermo Scientific) diluted (1:12,000) in PBST containing 3% (w/v) BSA for one hour at room temperature. Isotype-specific rabbit anti-chicken IgY was used to avoid potential cross-reactions with IgM. The color reaction was developed using OptEIA™ TMB substrate (BD) according to manufacturer's instructions. The reaction was terminated by addition of 1N sulfuric acid. Absorbances at 450 nm (A₄₅₀) were measured in a Wallac plate reader (PerkinElmer Inc., Waltham, Mass.).

The presence of peptide-specific IgG was determined by relating the mean A₄₅₀ value of each serum sample to that of a positive control serum sample (diluted at 1:100), which was used as the internal standard on all plates, to allow comparison of titers across plates and experiments, but within isotype. The relative levels of peptide-specific IgG in all serum samples were determined and normalized by calculating the sample to positive (S/P) ratio as follows: S/P value=(Sample mean−negative control mean)/(Positive control serum mean−negative control mean). The effect of specifically targeting the peptide to cCD40 (as opposed to incorporating it in a non-specific antibody complex) was estimated by using the following calculation: Mab 2C5 (S/P) minus MIg (S/P). Student's t-test was used to determine significant differences in means of S/P values between treatments across all groups, and S/P values of the MIg-peptide complex group were used as baseline. All data were analyzed and generated using JMP® version 9 software (SAS Institute Inc., Cary, N.C.). Statistical significance was determined at P<0.05.

Quantification of Peptide-Specific Tracheal sIgA by ELISA

Levels of peptide-specific sIgA in tracheal mucosa samples were determined by ELISA. Eight chickens from each croup were sacrificed at either seven or 14 days post immunization (p.i.), and the tracheal mucosa sample from each chick was collected by preparing a tracheal wash as follows. In order to avoid blood contamination of the trachea, every chicken was enthanized using a CO₂ chamber. The trachea was exposed aseptically at the pharyngeal region, and a 1-cm segment of trachea was collected, weighed, and then transferred to a 2-mL microcentrifuge tube. The trachea was suspended in cold PBST [137 mM NaCl, 2.7 mM KCl, 10 mM Na₂HPO₄, 2 mM KH₂PO₄, and 0.5% Tween 20 (v/v)] containing Halt® Protease and Phosphatase Inhibitor (Thermo Fisher Scientific Inc., Barrington, Ill.), 0.1% (w/v) thimerosal, and 3% (w/v) BSA. To maximize the extraction efficiency of tracheal IgA, 1 mL PBST was added per 100 mg trachea sample weight. The tracheal mucosa was sloughed off from the inner liner of the trachea by vigorously vortexing for 30 seconds. The tube was centrifuged at 5,000×g for 30 minutes at 4° C., and the supernatant was collected and frozen at −20° C. until use.

The detection of sIgA in the mucosal extracts was performed as follows. Biotinylated peptide (b-NAWSKEYARGFAKTGK; SEQ ID NO: 57) was incubated with goat anti-biotin antibody (Thermo Fisher Scientific Inc.) on a rotator at 37° C. for one hour. Flat-bottom, 96-well microtiter plates (Thermo Fisher Scientific Inc.) were coated with peptide-goat antibody complex (5 μg/mL) in 0.05M carbonate-bicarbonate buffer, pH 9.6 (SIGMA), overnight at 4° C. Excess peptide-goat antibody complex was removed, and plates were blocked with PBS, pH 7.4 containing 5% (w/v) bovine serum albumin (BSA) (Rockland Immunochemicals Inc., Gilbertsville, Pa.) overnight at 4° C. Peptide-coated wells were washed with PBST and then incubated with chicken tracheal IgA samples (diluted to 1:100) in PBST containing 3% (w/v) BSA overnight at 4° C. The plates were then washed as described above and incubated with horseradish peroxidase-conjugated goat anti-chicken IgA (Thermo Fisher Scientific Inc.) diluted (1:10,000) in PBST containing 3% (w/v) BSA for one hour at room temperature. Isotype-specific goat anti-chicken IgA was used to avoid the cross-reaction with other antibody isotypes. The color reaction was developed using OptEIA™ TMB substrate (BD, Lakes, N.J.) per the manufacturer's instructions, and terminated by addition of 1N sulfuric acid. Absorbances at 450 nm (A₄₅₀ were measured in a Wallac plate reader (PerkinElmer Inc., Waltham, Mass.). The presence of peptide-specific IgA was determined by relating the mean (A₄₅₀)value of each tracheal IgA sample to that of a positive control IgA sample used as internal standard (1:100). The relative levels of peptide-specific IgA in all tracheal samples were determined and normalized by calculating the sample to positive (S/P) ratio as explained above for IgG. Student's t-test was used to determine significant differences in means of S/P values between treatments across all groups, and S/P values of the MIg-peptide complex group were used as baseline. All data were analyzed and generated using JMP® version 9 software (SAS Institute Inc., Cary, N.C.). Statistical significance was determined at P<0.05.

### Results

Antibody Responses After a Single Parenteral (s.c.) Immunization with Anti-CD40-Guided Peptide Complex vs. Non-Specific, “Blind” Peptide Complex

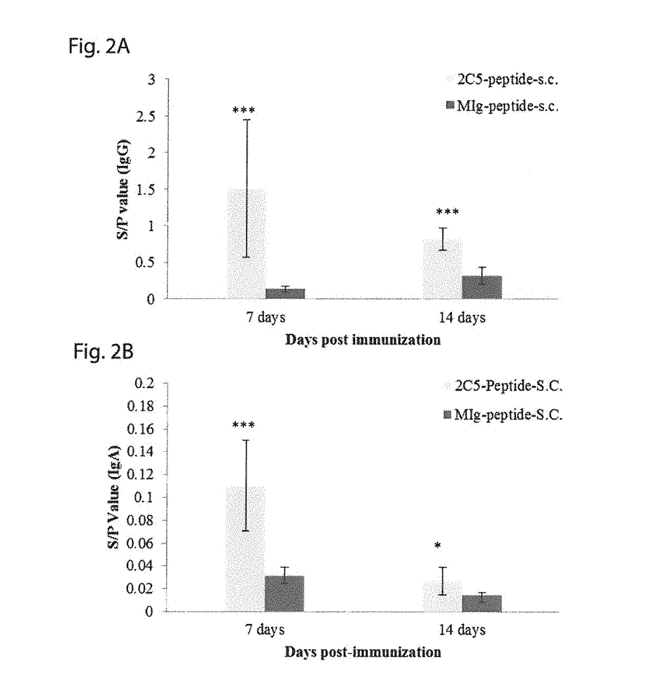

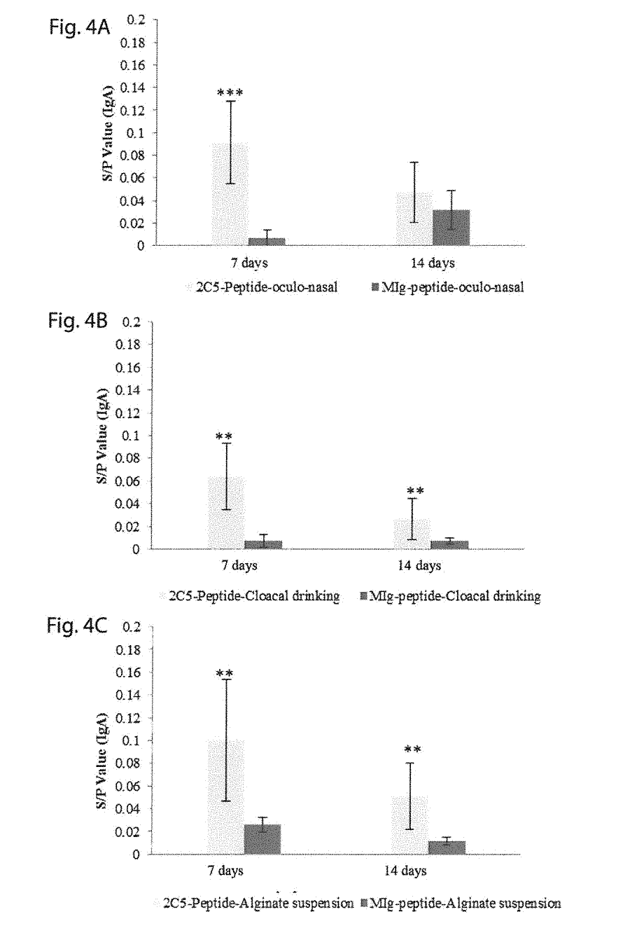

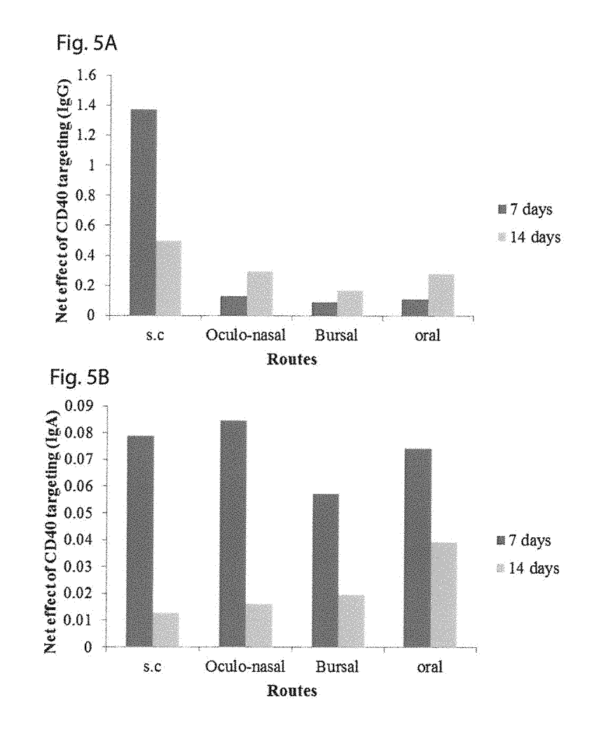

To evaluate the effect of parenteral immunization of anti-CD40-guided Mab 2C5-peptide complex on specific systemic and mucosal antibody responses, groups of five-week old male Leghorns received a single s.c. immunization with 50 μg Mab 2C5-peptide complex, and their responses were compared to those obtained with a “blind” non-specific MIg-peptide complex that served as the negative control. Trachea and plasma samples were collected from all immunized chickens at day 7 and 14 p.i. and peptide-specific IgA and IgG immune responses were assessed by ELISA. As shown in FIG. 2A, a single s.c. injection of Mab 2C5-peptide complex induced peptide-specific circulatory IgG antibody responses that were significantly higher than those obtained with non-specific MIg-peptide controls at 7 (P<0.001) and 14 days (P<0.001) p.i. Peptide-specific sIgA immune responses were also significantly enhanced on day 7 (P<0.001) and 14 (p<0.05) p.i. by targeting the immunogen to CD40 expressed on the chicken APCs (FIG. 2B). While we observed statistically significantly increased IgG and sIgA immune responses compared to controls on day 14 p.i., the major immune-enhancement was clearly observed on day 7 p.i. The same effect can also be observed, on the overview graph of all antibody responses shown in FIG. 4 and FIG. 5.

Antibody Responses After a Single Mucosal Immunization with Anti-CD40-Guided Peptide Complex vs. Non-Specific MIgG Peptide Complex

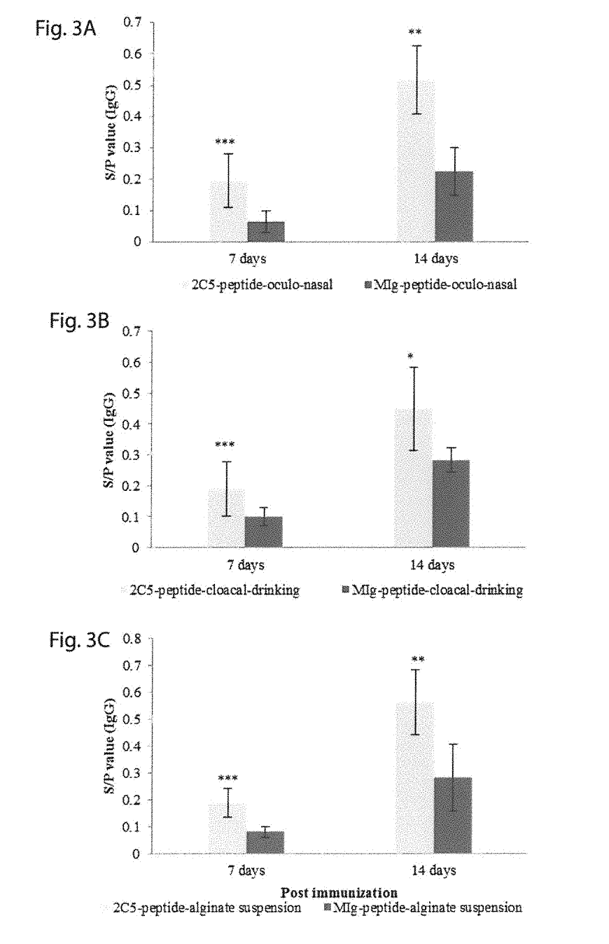

The potential immune-enhancing effect of the anti-CD40 Mab 2C5-peptide complex was also evaluated by administration of the immunogen via three different mucosal induction sites to the birds, each time using “blind” non-specific MIg-peptide complex as the negative control. Groups of five-week old male Leghorns were administrated a single Mab 2C5-peptide complex dose (50 μg) via one of the following mucosal routes: oculo-nasal (eye drops), cloacal-drinking (drops on the lips of the vent), and oral administration. The oral route was not administered by gavage into the stomach (which would bypass the esophagus and the crop) but active drinking of the immunogen solution. Trachea and plasma samples were collected 7 and 14 days p.i. and antibody responses were measured as described previously for the s.c. administration route. The results obtained from different mucosal routs of administration showed that 2C5-peptide complex induced similar antibody response patterns of IgG (FIG. 3) and sIgA (FIG. 4) for each of the different routes. Antigen directly delivered to mucosal inductive sites via all three mucosal routes induced significant peptide-specific systemic IgG immune responses from days 7 p.i. (P<0.001) onward through day 14 p.i. (oculo-nasal: P<0.001; oral: P<0.01; cloacal-drinking: P<0.05) compared to MIg-peptide control (FIG. 3). FIG. 4 shows that anti-CD40-guided Mab 2C5-peptide complex was also able to induce significant peptide specific sIgA responses through all three tested mucosal routes at days 7 p.i. (oculo-nasal: P<0.001; oral: P<0.01; cloacal-drinking: P<0.01) but those IgA responses clearly declined by day 14 p.i. (oculo-nasal: non-significant oral: P<0.01; cloacal-drinking: P<0.01) compared with MIg-peptide complex. Notably, mucosal administration of “blind” MIg-peptide complex through different routes also seemed to slightly numerically increase peptide-specific systemic IgG responses, and also the mucosal sIgA response but only after oculo-nasal administration.

### Calculation of the Net Immuno-Enhancing of Anti-CD40-Targeting Through Different Routes of Administration

The above results allow us to assess the net immuno-enhancing effect of targeting a peptide to CD40′ APCs, as opposed to incorporation of the same peptide in a non-specific, “blind” protein complex. For this purpose, the immuno-enhancing effect was defined as: [average (S/P) value of anti-CD40-guided complex) from which was subtracted [average (S/P) value of administration of “blind” complex]. This adjuvant effect was compared between administration routes (4) and time points (2).

As shown in FIG. 5A, s.c. administration of 2C5-peptide complex generated by far the most robust systemic IgG immune response achieved by CD40 targeting at day 7 p.i. However, the level of magnitude of this enhancement was not sustained and declined to less than half of the original value by day 14 p.i. (1.371 vs. 0.497). Although the net IgG effect of CD40 targeting through s.c. administration had declined by day 14 p.i., the net effect on systemic peptide-specific IgG levels was still higher than that obtained with any of the other mucosal routes, at any other time. The three mucosal administration routes posted similar but low net effect on systemic IgG responses at days 7 p.i. and moderately increased toward day 14 p.i. (FIG. 5A).

Surprisingly, s.c. immunization with 2C5-peptide complex induced a net effect of CD40 targeting on the secretion of peptide-specific IgA. The effect of the s.c. administration on specific IgA levels was similar in magnitude to that of the three different mucosal routes at day 7 p.i. (FIG. 5B). The net effect of CD40 targeting on peptide-specific IgA production had dropped substantially at day 14 p.i. in all routes of administration. This could be partially the result of the fact that by day 14 p.i., the blind MIg-peptide complex started slowly inducing sonic peptide-specific sIgA immune response, which detracts from the net CD40 -targeting effect of 2C5.

### Example 2

### Production of Anti-Chicken CD40 scFv

A single-chain antibody library (scFv) against chicken CD40 (chCD40) was constructed by phage display. Briefly, mice were immunized with chicken CD40 and splenocytes were collected. RNA was extracted and cDNA synthesized. The variable light and heavy chains were amplified using PCR and a scFv was amplified using PCR. The product was ligated into a vector and transformed into E. coli. After helper phage rescue the phage were precipitated. An scFv library size of 3×10 transformants was obtained. The phage library was added to a CD40-coated ELISA allowed to bind and washed to remove non-specifically bound phage. E. coli was added to allow amplification of bound phage and the process was repeated. Three rounds of panning against chicken CD40 resulted in a 40% enrichment of the positive clones, as those became the dominant population in the library as shown in Table 1 below.

TABLE 1
Panning to enrich for anti-CD40 scFv

| Round | Input | Output | % Bound (×10⁻⁴) | Enrichment |
|---|---|---|---|---|
| 1 | 7.2 × 10¹¹ | 5.7 × 10⁴ | 0.08 | |
| 2 | 6.2 × 10¹¹ | 8.8 × 10⁴ | 0.14 | 1.75 |
| 3 | 1.2 × 10¹² | 6.8 × 10⁶ | 5.7 | 40.7 |
| 4 | 7 × 10¹² | 1.5 × 10⁷ | 2.14 | |

% phage bound = (output/input) × 100.
Enrichment = fold increase of % phage bound compared to the previous round.

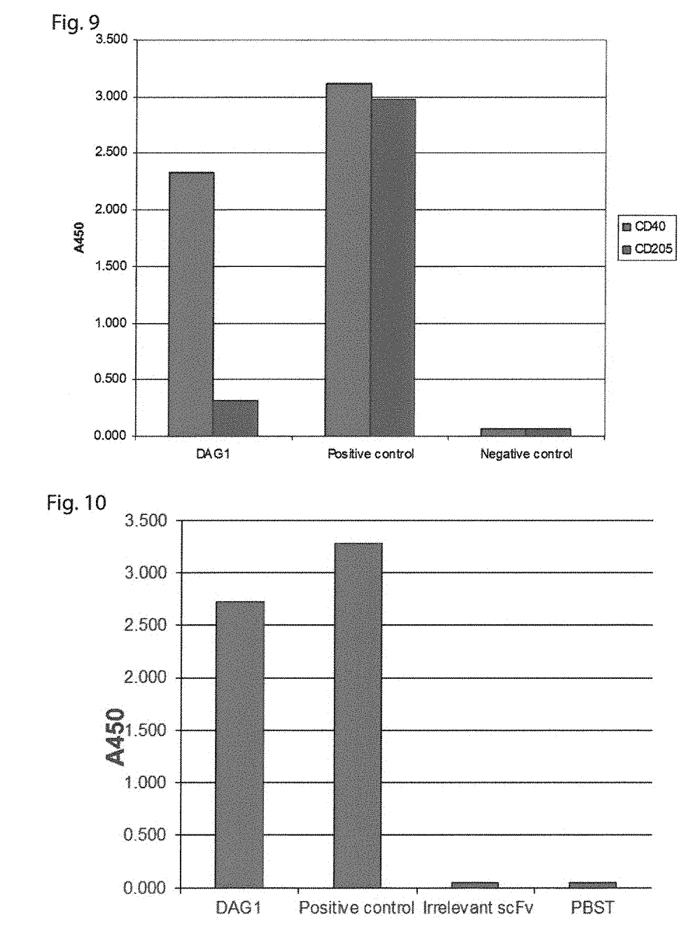

DAG1-displaying phage was then tested in an ELISA against cCD40 and CD205 and the results are shown in FIG. 9. See SEQ ID NO: 14. The scFv bound specifically to cCD40. Thus, following three rounds of panning against cCD40, specific, high-affinity antibodies were obtained. Soluble anti-cCD40 say designated DAG1 (˜35 KDa) was purified by nickel affinity chromatography and characterized by immunoblotting. This scFv recognized cCD40 in ELISA as shown in FIG. 10.

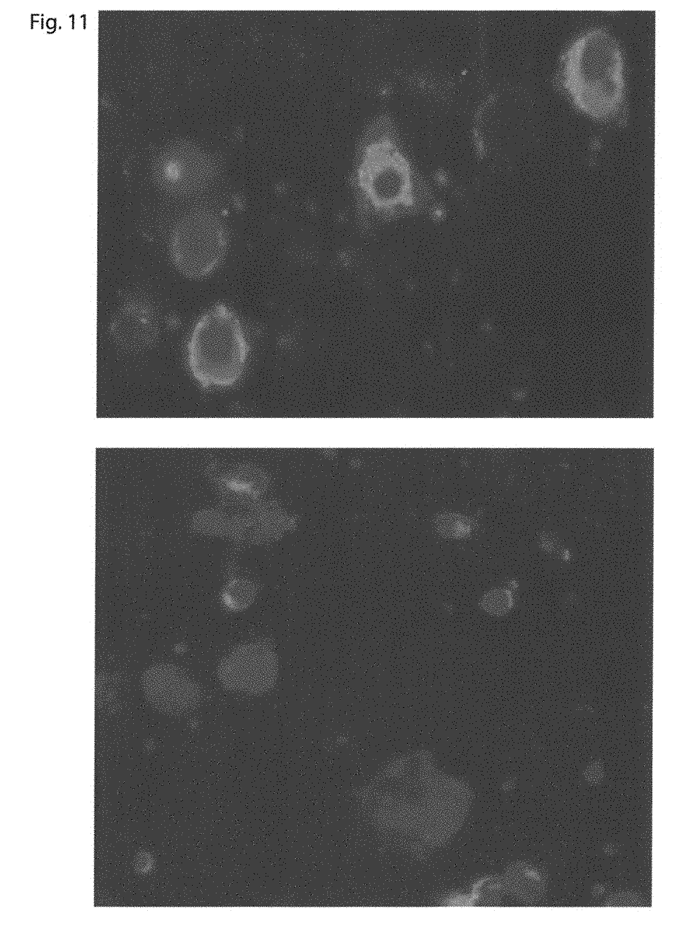

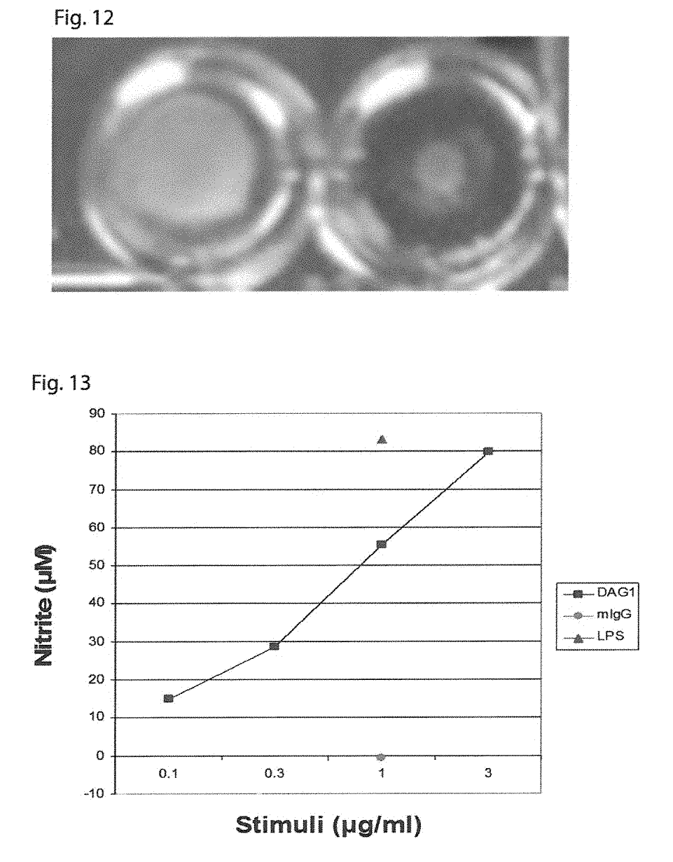

Cells (DT40 B cells or HD11 macrophages) were fixed on poly--L-lysine: coated slides using 4% paraformaldehyde iii PBS and stained with anti-cCD40 say DAG1. The DAG1 scFv was able to specifically bind to chicken CD40 expressed on chicken DT40 cells (FIG. 11A) and chicken HD11 macrophages (FIG. 11B). The ability of DAG1 scFv to agglutinate DT40 B cells in vitro was also tested. Cells (2×10⁵) were seeded in a V-bottom plate and were incubated overnight with either 10 μl of bacterial cell culture containing anti-cCD40 scFv (FIG. 12A) or with PBS (FIG. 12B). Cells incubated with DAG1 were agglutinated and formed a network on the well bottom and sides. Cells incubated with PBS collected into the V-bottom as shown in FIG. 12.

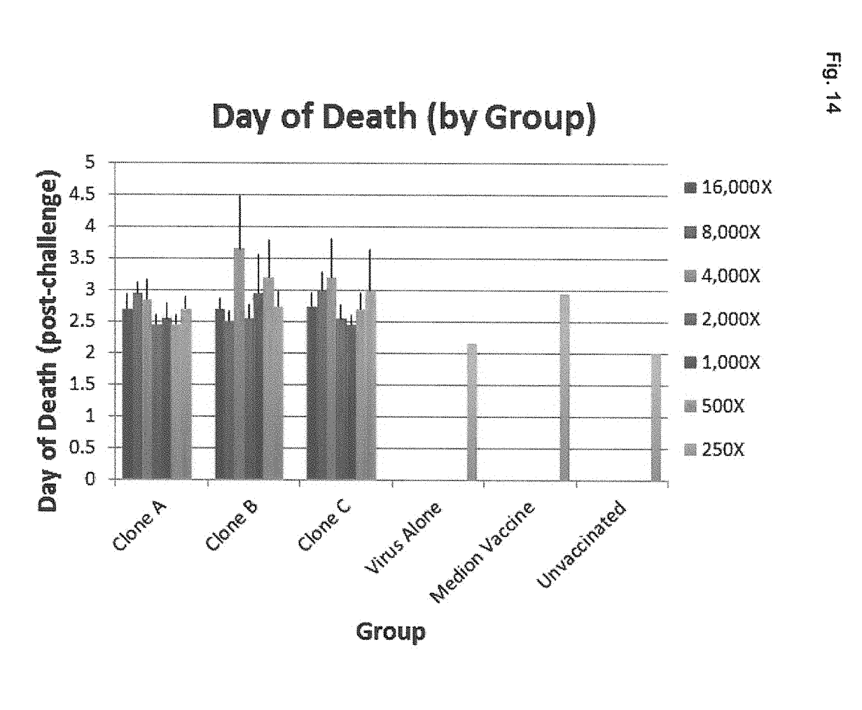

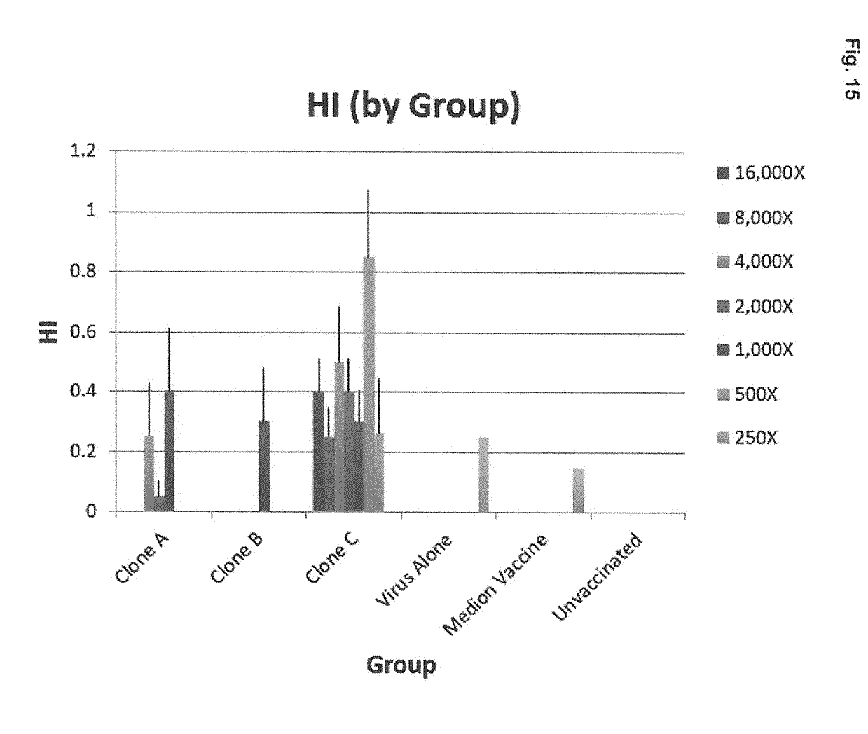

Nitric oxide production by HD11macrophages stimulated with serial three-fold dilutions of purified anti-cCD40 scFv (DAG1) (solid squares) mouse IgG1 (solid circle), or LPS (solid triangle) was assessed. As shown in FIG. 13, nitric oxide production was stimulated in a linear fashion in HD11 chicken macrophages when stimulated with dilutions of DAG1. These activities point to the ability of anti-cCD40 DAG1 to mimic the effects of CD40L (CD154), providing the signals needed to induce activation of chicken APCs in vitro. Such an agonistic anti-cCD40 scFv may therefore constitute a powerful tool to study the role of CD40 in the chicken immune system or be linked to antigens to induce immune responses.

### Example 3

### Avian Influenza Adjuvant Complex Testing

### Materials & Methods

Monoclonal antibodies were produced against the AIV conserved M2e ion channel domain. Based on previously published sequences, the M2e conserved peptide sequence of CEVETPTRN (SEQ ID NO: 58) was synthesized and used to immunize Balb/c mice subcutaneously at 50 μg/mouse in RIBI buffer. Three boosts of 25 μg/mouse subcutaneously were performed at three weeks intervals. Plasma was collected 1-week post each immunization to screen for peptide-specific IgG response based on ELISA. Once mice were hyper-immunized, antibody titers plateau, mice were euthanized and splenocytes harvested.

The splenocytes were used for electrofusion with mouse Sp2/0 myeloma cells to produce B-cell hybridomas. Hybridoma cultures were maintained at 37° C. at 5% CO₂ and cultured in DMEM media supplemented with 15% FBS. Hybridoma supernatants were screened for peptide-specific M2e antibody production via ELISA and ability to bind whole avian influenza virus. Parent hybridomas were chosen and subsequently subcloned by limiting dilution. Subcloned monoclonal hybridomas were screened yet again following the same methods before final subclones were chosen for ascites production and cryogenic storage. Three hybridomas were positive for whole avian influenza virus (AIV) recognitions (strongly positive), designated as Clone A, Clone B, and Clone C. These three subclones were used in the adjuvant complex formation and immunogenicity tests against AIV.

After ascites production, each of the three anti-M2e monoclonal antibodies chosen was purified by Protein G affinity chromatography and biotinylated using EZ Link Hydrazide LC Biotin kit from Thermo Scientific as per manufacturer's instructions. Biotinylated anti-M2e antibodies were complexed with biotinylated anti-CD40 monoclonal antibodies using streptavidin as a scaffold at a two first monoclonal antibody to one streptavidin to two second monoclonal antibody ratio. This anti-CD40/M2e complex was mixed with chemically inactivated whole avian influenza virus, previously propagated in embryonic chicken eggs, to allow binding of virus to the adjuvant complex. The completed complexes were used for in vivo immunogenicity studies in chickens at the Medion Vaccine Company in Bandung, Indonesia.

### Results

As shown in FIG. 14, the experimental adjuvants (from monoclonal M2e antibody clones A, B, and C) equally delayed death caused by HPAI challenge compared to the Mahon commercial vaccine control (by 1 day on average). All experimental groups had 384HA units of inactivated virus. Experimental groups had varying amounts of experimental adjuvant complex listed as amount of complex per viral particle. For example, 250× is 250 complexes per viral particle. The animals were challenged 1 week after vaccination with and H5 Avian influenza virus challenge at 2×10⁵ virus/bird. The unvaccinated group, as shown on the graph in FIG. 14, is the unvaccinated-challenged control group. The virus alone group received inactivated virus without adjuvant during vaccination.

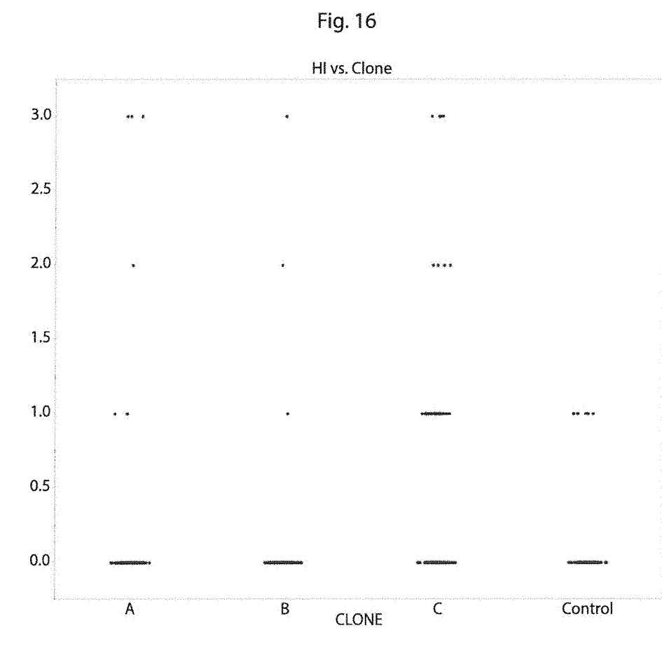

Sera were collected 1-week post-vaccination and used for HI testing (viral neutralization based on hemagglutination inhibition). Sera collected from birds were incubated with AIV to allow binding and neutralization of the virus. Whole red blood cells are added to verify if antibodies in sera were able to neutralize the virus' ability to hemagglutinate the red blood cells. Mean HI values per experimental adjuvant clone are shown in FIG. 15 and represent vaccine efficacy before challenge with HPAI. HI scores are widely established as accurately predictive for vaccine efficacy. While no statistical difference was observed within each group based on the ratio/dosage of adjuvant to viral particle, each of the M2e targeted complexes induced significant inhibition of hernaglutination. The experimental groups' HI were fully combined (disregarding ratios/dosages), and compared to the control as shown in FIG. 16. Distribution of mean HI values as shown in FIG. 16, in which each bird's response is an individual point in the graph, demonstrates that all experimental adjuvants induced higher HI values than the controls. Clone C shows the highest HI ability compared to Clone A or Clone B.

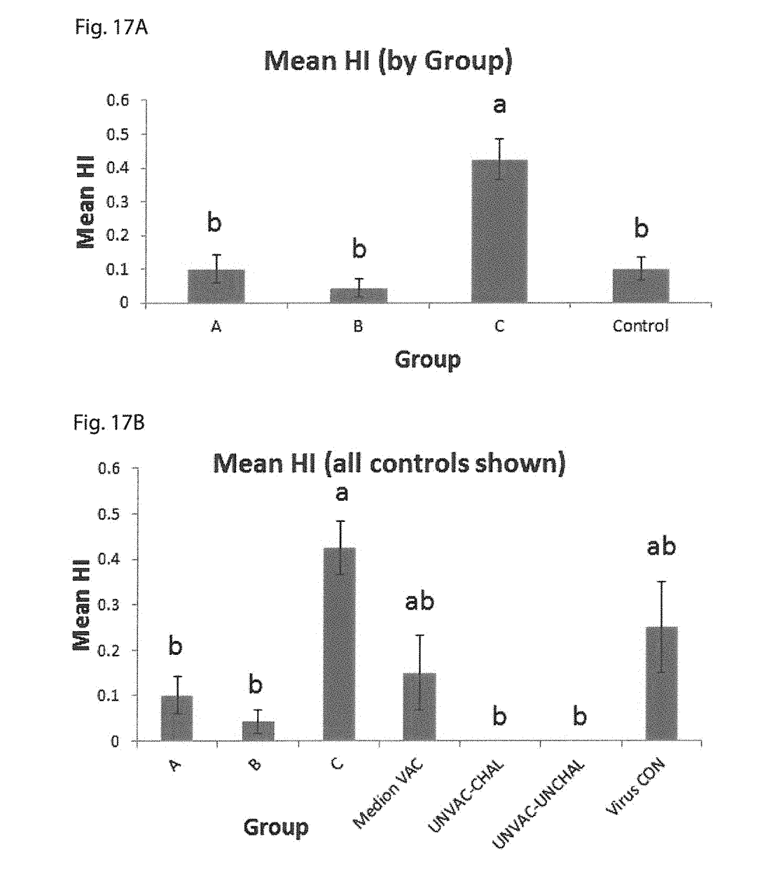

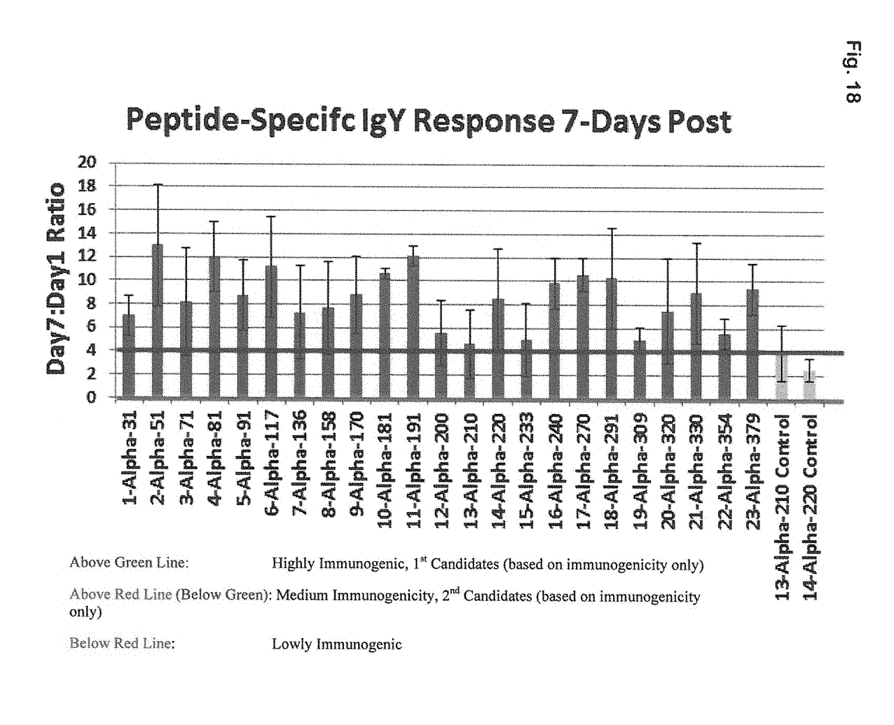

Statistically, Clone C shows values are significantly higher than the other groups (Clone A, Clone B, or the composited controls) as shown in FIG. 17A. If controls are separated (as in FIG. 17B), Clone C's score is not statistically, but only numerically higher than controls. It is important to remember that the Medion vaccine is a commercial vaccine control and thus any increase in performance is highly relevant. Clone C remains statistically higher than the other clones after control groups are separated. Overall, we have discovered that Clone C is clearly more effective than Clones A or B as a vaccine adjuvant. Adjuvant complex to viral particle ratio does not seem to be a major factor to inducing neutralizing antibody production (as seen in Clone C's HI data). The adjuvant complex is able to equally delay death after onset of HPAI infection, and has better HI titers than the commercial vaccine.

### Conclusion

The most important conclusion from this trial is that it delivers undeniable (statistical) proof for the theoretical tenet of the trial, i.e. that our adjuvant complex can physically link a chicken's antigen-presenting cells on one end with an inactivated AI viral particle at the other end, and provokes an incredibly fast immune response in the process. Until the in vivo trial, our initial concept was hypothesized using Avogadro's number to calculate the amount of adjuvant complex per routine dose of inactivated virus. The antibody-guided approach beat the Medion commercial vaccine.

### Example 4

### Antibody Guided C. perfringens α-Toxin Epitope Mapping

### Materials Methods

Extracellular domains of Clostridium perfringens alpha toxin were analyzed to identify possible regions for antibody neutralization of the toxin's hemolytic activity. A library of linear peptides of 8-15 amino acids each in length was chosen based on their potential as B-cell epitopes and synthesized. See Table 2 and SEQ ID NOs: 59-83.

Each biotinylated peptide from the epitope library was incorporated into the CD40-targeting complex (biotinylated peptide linked via streptavidin to the biotinylated CD40 antibody) and subcutaneously injected into birds to induce peptide-specific IgG antibody responses. CD40 antibody was biotinylated using commercial biotinylation kits (EZ Link Hydrazide LC Biotin from Thermo Scientific) and peptides were purchase already biotinylated. Antiserum was collected from each bird 1-week-post-immunization. After serum collection, samples were centrifuged to remove debris and precipitates. Peptide-specific immunogenicity was measured by standardized ELISA protocols.

Antiserum produced against each target was tested for its ability to neutralize hemolytic activity. C. perfringens alpha toxin was obtained from the USDA. Fifty microliters of toxin at 1:80 dilution (USDA suggested toxin dilution for neutralization assays) in sterile PBS was mixed with 50 μL of serum (2-fold serial dilution of serum starting from 1:10) on a flat-bottom 96-well plate and incubated at 37° C. for 1 hour to allow binding/neutralization of the toxin. After initial incubation, 100 μL of 5% (v/v) sheep red blood cells in PBS was added to all wells and incubated for another hour at 37° C. After incubation, neutralization of hemolytic activity was observed in the wells.

TABLE 2

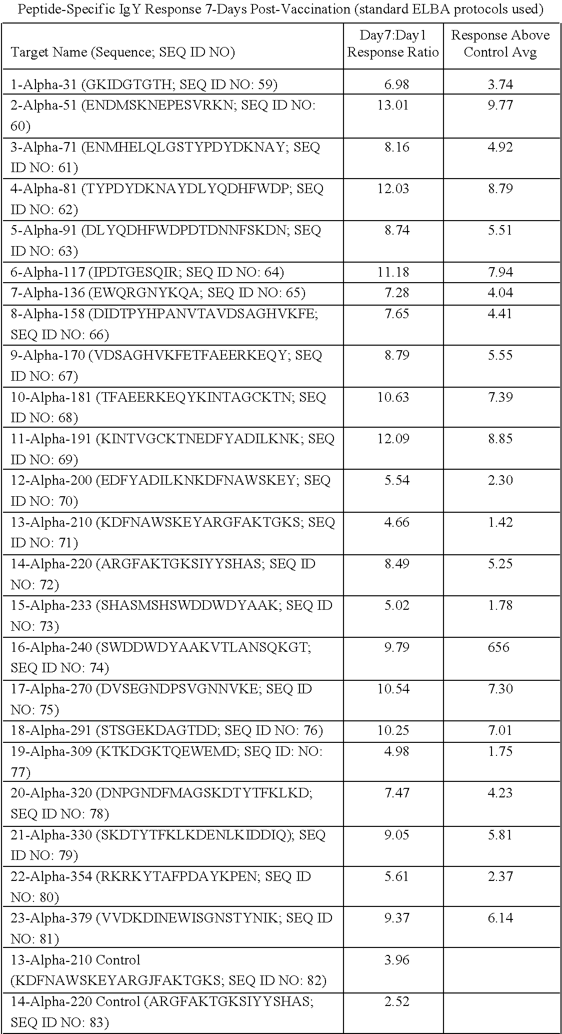

The data showing the antibody response in graphic form are displayed in FIG. 18. The antibody responses were broken into three groups. Those with a 7 day after immunization to day of immunization ratio of peptide specific immunoglobulin over 10 were considered highly immunogenic. The peptide complexes with ratios between 6 and 10 were considered moderately immunogenic and those with ratios of less than 6 were considered mildly immunogenic. These distinctions are shown graphically as the lines across the graph in FIG. 18.

A viral neutralization assay was then completed to determine if the antibodies were capable of neutralizing the hemolytic activity of the Clostridium perfringens alpha toxin. Briefly, two-fold serial dilutions of the sera were made in saline and 50 μL added per well. A 1:80 dilution of the C. perfringens alpha toxin obtained from the USDA was prepared in sterile PBS and added at 50 μL per well. The assay was incubated for 1 hour at 37° C. Then 100 μL of a 5% sheep red blood cell suspension was added to each well, mixed gently and allowed to incubate for 1 hour at 37°C. The absorbance at 490 nm was measured to determine the level of hemolysis of the red blood cells. Wells positive for hemolysis were sera that were considered negative for neutralization and vice versa.

As shown in Table 3 below, several of the sera were able to neutralize the toxin and prevent hemolysis. The neutralization reported in the Table is the highest dilution factor still capable of neutralizing C. perfringens alpha toxin. So “160” means serum still neutralized the toxin at 1:160 dilution. Control Peptides (non-guided system used) were negative for hemolytic neutralization.

Antibodies generated one week after a single injection with CD-40-targeted antibody guided antigens, resulted in some degree of diminution of alpha-toxin hemolytic activity. This vaccination technique, with antibody-guided antigens, resulted in significant immune response (measured as IgY levels) in 9/23 antigens. Additionally, through this antigen selection process, epitopes 20, 21, and 23 were both highly immunogenic and highly neutralizing for hemolytic activity, suggesting their potential as vaccine candidates. Thus, we have developed a rapid method to map epitopes and identify potential antigenic epitopes for use in recombinant vaccine generation.

1. An adjuvant composition comprising at least one first CD40 agonistic antibody or portion thereof comprising at least two F(ab) regions capable of specifically binding CD40 and inducing CD40 signaling, at least one second antibody or portion thereof comprising at least two F(ab) regions capable of specifically binding a microorganism, at least one label attached to the at least one first CD40 agonistic antibody or portion thereof and the at least one second antibody or portion thereof, and a linker moiety capable of specifically binding to the labels, wherein the at least one first CD40 agonistic antibody and the at least one second antibody are bound to the linker moiety to form a complex.

2. (canceled)

3. The adjuvant composition of claim 1, wherein two or more of the first CD40 agonistic antibody and two or more of the second antibody are bound to the linker moiety to form the complex.

4. (canceled)

5. The adjuvant composition of claim 1, wherein the label on each of the first CD40 agonistic antibody and the second antibody is biotin.

6. The adjuvant composition of claim 1, wherein the linker moiety is avidin or streptavidin.

7. The adjuvant composition of claim 1, wherein the microorganism to which the second antibody specifically binds is a bacterium or a virus.

8. The adjuvant composition of claim 7, wherein the second antibody specifically binds a microorganism selected from the group consisting of influenza virus, Salmonella, Clostridium, Campylobacter, Escherichia, Shigella, Helicobacter, Vibrio, Plesiomonas, Edwardia, Clostridia, Klebsiella, Staphylococcus, Streptococcus, Aeromonas, Foot and Mouth virus, porcine epidemic diarrhea virus (PEDv), and Porcine reproductive and respiratory syndrome virus (PRRSV).

9. The adjuvant composition of claim 8, wherein the second antibody binds Influenza M2e.

10. The adjuvant composition of claim 1, wherein the first CD40 agonistic antibody or portion thereof is selected from the group consisting of at least one of:
a. An antibody comprised of SEQ ID NO: 2 and SEQ ID NO: 4 (2C5);
b. An antibody comprised of SEQ ID NO: 14 (DAG1);
c. An antibody or portion thereof comprising a heavy chain variable (V_{H}) region and a light chain variable (V_{L}) region, wherein the heavy chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7 and wherein the light chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and
d. An antibody or portion thereof comprising a heavy chain variable (V_{H}) region and a light chain variable (V_{L}) region, wherein the heavy chain variable region comprises a CDR1 comprising the amino acid sequence set forth in SEQ ID NO:
20, a CDR2 comprising the amino acid sequence set forth in SEQ ID NO: 21, and a CDR3 comprising the amino acid sequence set forth in SEQ ID NO: 22 and wherein the light chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19.

11. (canceled)

12. A vaccine comprising the adjuvant composition of claim 1 and further comprising the microorganism, wherein the adjuvant composition is specifically bound to the microorganism.

13. (canceled)

14. The vaccine of claim 12, wherein the microorganism is killed or inactivated.

15. The vaccine of claims 12, wherein the vaccine is comprised within alginate spheres.

16. (canceled)

17. A CD40 agonistic antibody or a portion thereof comprising at least an F(ab) region, the CD40 agonistic antibody or portion thereof selected from the group consisting of at least one of:
a. An antibody comprised of SEQ ID NO: 2 and SEQ ID NO: 4 (2C5);
b. An antibody comprising SEQ ID NO: 14 (DAG1);
c. An antibody or portion thereof comprising a heavy chain variable (V_{H}) region and a light chain variable (V_{L}) region, wherein the heavy chain variable region comprises a CDR1 comprising the amino acid sequence set forth in SEQ ID NO: 5, a CDR2 comprising the amino acid sequence set forth in SEQ ID NO: 6, and a CDR3 comprising the amino acid sequence set forth in SEQ ID NO: 7 and wherein the light chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10 (2C5); and
d. An antibody or portion thereof comprising a heavy chain variable (V_{H}) region and a light chain variable (V_{L}) region, wherein the heavy chain variable region comprises a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22 and wherein the light chain variable region comprises a CDR1 comprising the amino acid sequence set forth in SEQ ID NO: 17, a CDR2 comprising the amino acid sequence set forth in SEQ ID NO: 18, and a CDR3 comprising the amino acid sequence set forth in SEQ ID NO: 19 (DAG1).

18. A vaccine comprising the CD40 agonistic antibody or portion thereof of claim 17 linked to an antigen by a linker moiety.

19. The vaccine of claim 18, wherein the linker moiety is selected from the group consisting of a peptide and streptavidin and wherein when the linker moiety is streptavidin, the CD40 agonistic antibody is biotinylated and the antigen is biotinylated such that the linker moiety is capable of linking the CD40 agonistic antibody to the antigen.

20. (canceled)

21. The vaccine of claim 18, wherein the antigen is selected from the group consisting of a vaccine, an influenza virus, a microorganism, a peptide, Salmonella, Clostridium perfringens, Campylobacter, Escherichia, Shigella, Helicobacter, Vibrio, Plesiomonas, Edwardia, Clostridia, Klebsiella, Staphylococcus, Streptococcus, Aeromonas, Foot and Mouth virus, porcine epidemic diarrhea virus (PEDv), and Porcine reproductive and respiratory syndrome virus (PRRSV).

22.-25. (canceled)

26. The vaccine of claim 18, wherein the vaccine is comprised within alginate spheres.

27. A pharmaceutical composition comprising the vaccine of claim 12 and a pharmaceutically acceptable carrier.

28. A method of enhancing an immune response in a subject comprising administering the vaccine of claim 12 to the subject in an amount effective to enhance the immune response to the antigen or microorganism.

29. The method of claim 28, wherein administration is via a route selected from the group consisting of mucosal oral, cloacal, nasal, ocular, subcutaneous route, in the food and in the drinking water.

30.-35. (canceled)

36. The method of claim 35, wherein the CD40 antibody is specific for chicken CD40 and the subject is a chicken.

37. A construct comprising a first polynucleotide encoding a CD40 agonistic antibody heavy chain comprising SEQ ID NO: 5, 6, and 7 or SEQ ID NO: 20, 21 and 22 and a CD40 agonistic antibody light chain comprising SEQ ID NO: 8, 9, and 10 or SEQ ID NO: 17, 18 and 19 and wherein the first polynucleotide is operably connected to a promoter to allow for expression of the CD40 agonistic antibody.

38. (canceled)

39. (canceled)

40. The construct of claim 37, further comprising a second polynucleotide encoding an antigen.

41. The construct of claim 40, wherein the antigen is selected from SEQ ID NOs: 26-53 or 57-83.

42. (canceled)

43. A cell comprising the construct of claim 37.

44. (canceled)

45. A method of epitope mapping a polypeptide comprising:
a. Generating labeled peptides of 8-20 amino acids from the polypeptide;
b. Attaching the labeled peptides to a labeled CD40 antibody via a linker moiety to create a CD40 antibody-peptide complex;
c. Administering the CD40 antibody-peptide complex to a subject;
d. Collecting sera from the subject;
e. Testing the sera for the presence of antibodies able to recognize the polypeptide; and
f. Identifying the peptides capable of producing antibodies to the polypeptide as antigenic epitopes.

46.-49. (canceled)
